(19) United States
(12) Patent Application Publication
(10) Pub. No.: US 2025/0360087 A1
(43) Pub. Date: Nov. 27, 2025

(54) COMPOSITIONS COMPRISING LIPID DROPLETS ENCAPSULATED WITHIN POLYSACCHARIDE WALLS AND USES THEREOF

(71) Applicant: UNIVERSITY OF SOUTH AUSTRALIA, Adelaide (AU)

(72) Inventors: Paul Matthew JOYCE, Adelaide (AU); Clive Allan PRESTIDGE, Semaphore South (AU)

(21) Appl. No.: 18/873,222

(22) PCT Filed: Jun. 9, 2023
(86) PCT No.: PCT/AU2023/050514

§ 371 Date: Dec. 9, 2024

(30) Foreign Application Priority Data
Jun. 10, 2022 (AU) 2022901597

(51) Int. Cl. A61K 9/50; A61K 9/107; A61K 31/216; A61K 31/496; A61K 31/733; A61P 3/04

(52) U.S. Cl. CPC A61K 9/5036; A61K 9/1075; A61K 31/216; A61K 31/496; A61K 31/733; A61P 3/04

(57) ABSTRACT

Drug delivery systems are needed to assist in improving the therapeutic characteristics of pharmaceutical agents. Provided is a dry composition, comprising a polysaccharide, such as inulin, and lipid droplets, wherein the lipid droplets are encapsulated within polymeric chains of the polysaccharide, and wherein the polysaccharide is not in a nano-particulate form. The use of such compositions enables efficient delivery of agents, such as poorly-water soluble drugs and antibiotics.

### PRIORITY CLAIM

This application claims priority from Australian provisional patent application number 2022901597 filed on 10 Jun. 2022, the content of which is to be taken as incorporated herein by this reference.

### FIELD OF THE INVENTION

The present invention relates generally to compositions which are effective carriers for the delivery of active substances to a subject. The compositions can be used for improving the health of a subject, or when comprising an active substance can be used for treating or preventing a disease or disorder in a subject, or for treating or preventing microbial infections.

### BACKGROUND OF THE INVENTION

The ability to effectively deliver active substances, such as pharmaceutical agents, to achieve a desired therapeutic effect in subjects is challenging. Principles related to the physical and chemical characteristics of the agent, agent preparation and formulation, route of administration, site-specific targeting, and metabolism, all play a part in ultimate delivery and treatment efficacy. Importantly, effective agent delivery rests in large part to the engineering of delivery systems which cater for shortcomings in agent characteristics. Indeed, research into new delivery systems has been progressing, as opposed to new drug development which has been declining, with one of the driving factors being the high cost of developing new drugs.

The effective delivery of poorly water-soluble active substances is particularly challenging. While the preferred route of drug administration is the oral route, about 90% of new orally administered drugs are poorly water-soluble lipophilic drugs. This results in precipitation of the crystalline drug due to the low lipophilic environment in the gastrointestinal tract, consequently reducing absorption and bioavailability. Poorly soluble drugs intended for parenteral delivery generally have to be solubilised with large amounts of co-solvents and surfactants, often resulting in adverse physiological reactions. Ocular delivery of poorly soluble drugs is challenging due to the absorption barriers and clearance mechanisms. Poorly soluble drugs administered nasally are limited by a relatively small administered volume, the geometry of the nasal cavity, and the strict safety requirements of the excipients used in the formulation. Finally, successful formulation design of poorly soluble drugs intended for pulmonary administration is hindered by the limited number of excipients generally recognized as safe for this route of delivery and the anatomical and physiological clearance mechanisms found in the airways.

The effective delivery of antimicrobial agents to subjects also faces challenges. An antimicrobial is an agent that kills, or inhibits the growth of, microorganisms. Antimicrobials can be classified based on the microorganism they primarily act against. For example, antibacterial agents such as antibiotics target bacteria, whereas antifungal agents are used against fungi. Since the discovery of penicillin in 1928, and its subsequent purification and development as an antibacterial agent, antibiotics have underpinned modern medicine. In fact, their use has been indispensable for the treatment of serious infections such as tuberculosis, meningitis and pneumonia, for preventing surgical site infections, and for managing immunocompromised individuals.

The efficacy of antimicrobial agents in subjects is hampered by ineffective delivery to the site of infection as a result of factors such as an inability to cross membranes of cells hosting pathogens, and the presence of biofilms associated with microbial infections. Intracellular pathogens serve as a problematic source of infection, and therefore effective treatment, due to their ability to evade biological immune responses. Intracellular pathogens shelter and live mostly within the mononuclear phagocyte system (MPS), comprised of phagocytic cells, such as blood monocytes and tissue macrophages. The main role of the MPS is to clean the blood stream by engulfing and killing foreign particles/micro-organisms by forming phagolysosomes that digest foreign particles/micro-organisms. However, intracellular bacteria are capable of ‘hijacking’ the signalling pathway in order to live in the environment of a host cell and hence use macrophages as a sanctuary. This reservoir allows pathogens to establish secondary infectious foci and leads to the recurrence of systemic infections.

This is further exacerbated by the inability for conventional antibiotics to efficiently penetrate cellular membranes. Indeed, antibacterial agents belonging to the β-lactam and aminoglycoside families have limited penetration to the host cells due to their high hydrophilicity. Furthermore, fluoroquinolones and macrolides exhibit restricted and relatively low intracellular retention, despite their ability to penetrate rapidly across cellular membranes. To compensate for the low drug concentration at the target site, high doses of antibiotics are frequently prescribed, which further contributes to antibiotic resistance. Despite scientific advancements, less than one-third of prescribed antibiotics exert any activity against intracellular pathogens. Currently only 19 new antimicrobial compounds have progressed to the final stages of clinical trials, where there is no guarantee that these conventional treatments will lead to improved bacterial eradication. Thus, it is expected that bacteria will continuing acquiring resistance unless alternative strategies to antimicrobial molecules and formulations are improved.

Furthermore, the presence of bacteria in biofilms significantly reduces, and often eliminates, the ability of antibacterial agents to exert their intended effect. Bacterial biofilms are linked to more than 60% of chronic infections and have been attributed to more than half of a million deaths globally each year. For example, biofilms have been implicated in common infectious processes such as bacterial vaginosis, urinary tract infections, catheter infections, middle-ear infections, formation of dental plaque, gingivitis, and coating contact lenses. The involvement of biofilms in less common, but more lethal processes, include endocarditis, infections in cystic fibrosis, and infections of permanent indwelling devices such as joint prostheses, catheters, heart valves, and intervertebral discs. For example, over half of the five million central venous catheters placed each year will develop a biofilm infection, despite the advances in clinical approaches. Furthermore, bacterial biofilms can impair cutaneous wound healing and reduce topical antibacterial efficiency in healing or treating infected skin wounds.

Bacteria within a biofilm are surrounded by a thick matrix of extracellular polymeric substances (EPS), including proteins, DNA, polysaccharides and lipids. The EPS protects the bacteria from the outside environment and enables a closely packed community that are in continuous communication through quorum sensing. Biofilm microbial eradication relies heavily on conventional antimicrobial agents which frequently fail to be efficient treatments due to various limitations including poor solubility and permeability through the biofilm matrix and the bacterial cell membranes. The EPS effectively acts as a protective mechanism for the bacteria, which significantly increases the tolerance to antibiotics.

In light of the issues above, there is a clear need for the development of compositions which enable the effective delivery of active substances such as poorly water-soluble drugs and antibiotics to subjects so as to maximise their therapeutic potential.

The discussion of documents, acts, materials, devices, articles and the like is included in this specification solely for the purpose of providing a context for the present invention. It is not suggested or represented that any or all of these matters formed part of the prior art base or were common general knowledge in the field relevant to the present invention as it existed before the priority date of each claim of this application.

### SUMMARY OF THE INVENTION

The present invention is predicated in part on the identification of a dry composition which provides significant and unexpected advantages with respect to the delivery of active substances to subjects in need thereof. The dry composition encompassed by the present invention enables efficient delivery of agents such as poorly water-soluble drugs to a subject, and enables delivery of agents to microbial cells and biofilms. Furthermore, when administered alone to a subject the dry composition encompassed by the present invention also provides a health benefit, including a gastrointestinal health benefit, to the subject.

Accordingly, in a first aspect the present invention provides a dry composition comprising:
(i) a polysaccharide; and
(ii) lipid droplets,
wherein the lipid droplets are encapsulated within polymeric chains of the polysaccharide, and wherein the polysaccharide is not in a nano-particulate form.

In some embodiments, the polysaccharide is a dietary polysaccharide. In some embodiments, the polysaccharide is selected from the group consisting of inulin, cellulose and glucomannan.

In some embodiments, the lipid droplets comprise a medium chain length fatty acid. In some embodiments, the ratio of polysaccharide to lipid in the composition ranges from about 10:90 to about 90:10. In some embodiments, the ratio of polysaccharide to lipid in the composition ranges from about 50:50 to about 75:25.

In some embodiments, the composition is used for improving the health of a subject. In some embodiments, the gastrointestinal health of the subject is improved.

In some embodiments, the composition further comprises an active substance, wherein the active substance is contained within the lipid droplets. In some embodiments, the active substance is an agent that can dissolve in the lipid droplets. In some embodiments, the active substance is a pharmaceutical agent.

In some embodiments, the pharmaceutical agent is a poorly water-soluble drug. In some embodiments, the pharmaceutical agent is selected from the group consisting of an antimicrobial agent, an anti-inflammatory agent, an anti-histamine, a cholesterol-lowering drug, or a psychotropic drug.

In some embodiments, the antimicrobial agent is one or more of an antibiotic, an antimicrobial peptide, and an antifungal agent. In some embodiments, the antibiotic is selected from the group consisting of rifampicin, tobramycin and vancomycin. In some embodiments, the cholesterol-lowering drug is a statin or fenofibrate. In some embodiments, the statin is simvastatin. In some embodiments, the psychotropic drug is lurasidone.

In some embodiments, the composition further comprises an excipient or stabilizer. In some embodiments, the stabilizer is lecithin.

In some embodiments, the composition is formulated for oral delivery.

In some embodiments, the composition is produced by a method comprising:
(i) producing a lipid-in-water nano-emulsion comprising the lipid droplets and the polysaccharide, wherein the polysaccharide is dissolved in the aqueous phase of the nano-emulsion; and
(ii) spray-drying the nano-emulsion.

In some embodiments, the nano-emulsion is produced by homogenizing a mixture comprising the lipid droplets and adding the polysaccharide in aqueous form to the mixture.

In some embodiments, the microparticles have an average diameter of <15 μm.

In a second aspect, the present invention provides a method for improving the health of a subject, or for treating or preventing a disease or disorder in a subject, the method comprising administering the composition of the first aspect of the invention to the subject.

In some embodiments, the gastrointestinal health of the subject is improved.

In some embodiments, a metabolic disease or disorder is treated or prevented in the subject.

In a third aspect, the present invention provides a method for treating or preventing a microbial infection is a subject, the method comprising administering the composition of the first aspect of the invention to the subject, wherein the pharmaceutical agent is an antimicrobial agent.

In some embodiments, the microbial infection is a bacterial infection. In some embodiments, the bacterial infection is due to Staphylococcus aureus. In some embodiments, the bacterial infection forms part of a biofilm.

In a fourth aspect, the present invention provides a method for administering an active substance to a subject, wherein the method comprises administering to the subject a composition of the first aspect of the invention.

In a fifth aspect, the present invention provides a method for producing a composition of the first aspect of the invention, wherein the method comprises spray-drying a lipid-in-water nano-emulsion comprising the lipid droplets and the polysaccharide.

In a sixth aspect, the present invention provides a dry composition comprising:
(i) a polysaccharide;
(ii) lipid droplets; and
(iii) an excipient or stabilizer,
wherein the lipid droplets are encapsulated within polymeric chains of the polysaccharide, and wherein the polysaccharide is not in a nano-particulate form.

In some embodiments, the polysaccharide is a dietary polysaccharide. In some embodiments, the polysaccharide is selected from the group consisting of inulin, cellulose and glucomannan.

In some embodiments of the sixth aspect of the invention, the lipid droplets comprise a medium chain length fatty acid. In some embodiments, the ratio of polysaccharide to lipid in the composition ranges from about 10:90 to about 90:10. In some embodiments, the ratio of polysaccharide to lipid in the composition ranges from about 50:50 to about 75:25.

In some embodiments of the sixth aspect of the invention, the stabilizer is lecithin.

In some embodiments of the sixth aspect of the invention, the composition is used for improving the health of a subject. In some embodiments, the gastrointestinal health of the subject is improved.

In some embodiments of the sixth aspect of the invention, the composition further comprises an active substance, wherein the active substance is contained within the lipid droplets. In some embodiments, the active substance is an agent that can dissolve in the lipid droplets. In some embodiments, the active substance is a pharmaceutical agent.

In some embodiments of the sixth aspect of the invention, the pharmaceutical agent is a poorly water-soluble drug. In some embodiments, the pharmaceutical agent is selected from the group consisting of an antimicrobial agent, an anti-inflammatory agent, an anti-histamine, a cholesterol-lowering drug, or a psychotropic drug.

In some embodiments of the sixth aspect of the invention, the antimicrobial agent is one or more of an antibiotic, an antimicrobial peptide, and an antifungal agent. In some embodiments, the antibiotic is selected from the group consisting of rifampicin, tobramycin and vancomycin. In some embodiments, the cholesterol-lowering drug is a statin or fenofibrate. In some embodiments, the statin is simvastatin. In some embodiments, the psychotropic drug is lurasidone.

In some embodiments of the sixth aspect of the invention, the composition is produced by a method comprising:
(i) producing a lipid-in-water nano-emulsion comprising the lipid droplets, the polysaccharide, and the excipient or stabilizer, wherein the polysaccharide is dissolved in the aqueous phase of the nano-emulsion; and
(ii) spray-drying the nano-emulsion.

In some embodiments of the sixth aspect of the invention, the nano-emulsion is produced by homogenizing a mixture comprising the lipid droplets and the excipient or stabilizer, and adding the polysaccharide in aqueous form to the mixture.

In some embodiments of the sixth aspect of the invention, the microparticles have an average diameter of <15 μm.

In a seventh aspect, the present invention provides a method for improving the health of a subject, or for treating or preventing a disease or disorder in a subject, the method comprising administering the composition of the sixth aspect of the invention to the subject.

In some embodiments, the gastrointestinal health of the subject is improved.

In some embodiments of the seventh aspect of the invention, a metabolic disease or disorder is treated or prevented in the subject.

In an eighth aspect, the present invention provides a method for treating or preventing a microbial infection is a subject, the method comprising administering the composition of the sixth aspect of the invention to the subject, wherein the pharmaceutical agent is an antimicrobial agent.

In some embodiments, the microbial infection is a bacterial infection. In some embodiments, the bacterial infection is due to Staphylococcus aureus. In some embodiments, the bacterial infection forms part of a biofilm.

In a ninth aspect, the present invention provides a method for administering an active substance to a subject, wherein the method comprises administering to the subject a composition of the sixth aspect of the invention.

In a tenth aspect, the present invention provides a method for producing a composition of the sixth aspect of the invention, wherein the method comprises spray-drying a lipid-in-water nano-emulsion comprising the lipid droplets, the polysaccharide, and the excipient or stabilizer.

In an eleventh aspect, the present invention provides a dry composition comprising:
(i) inulin;
(ii) an active substance; and
(iii) lipid droplets,
wherein the lipid droplets are encapsulated within polymeric chains of inulin, and wherein the polysaccharide is not in a nano-particulate form.

In some embodiments, the lipid droplets comprise a medium chain length fatty acid. In some embodiments, the ratio of polysaccharide to lipid in the composition ranges from about 10:90 to about 90:10. In some embodiments, the ratio of polysaccharide to lipid in the composition ranges from about 50:50 to about 75:25.

In some embodiments of the eleventh aspect of the invention, the active substance is contained within the lipid droplets. In some embodiments, the active substance is an agent that can dissolve in the lipid droplets. In some embodiments, the active substance is a pharmaceutical agent.

In some embodiments of the eleventh aspect of the invention, the pharmaceutical agent is a poorly water-soluble drug. In some embodiments, the pharmaceutical agent is selected from the group consisting of an antimicrobial agent, an anti-inflammatory agent, an anti-histamine, a cholesterol-lowering drug, or a psychotropic drug.

In some embodiments of the eleventh aspect of the invention, the antimicrobial agent is one or more of an antibiotic, an antimicrobial peptide, and an antifungal agent. In some embodiments, the antibiotic is selected from the group consisting of rifampicin, tobramycin and vancomycin. In some embodiments, the cholesterol-lowering drug is a statin or fenofibrate. In some embodiments, the statin is simvastatin. In some embodiments, the psychotropic drug is lurasidone.

In some embodiments of the eleventh aspect of the invention, the composition further comprises an excipient or stabilizer. In some embodiments, the stabilizer is lecithin.

In some embodiments of the eleventh aspect of the invention, the composition is formulated for oral delivery.

In some embodiments of the eleventh aspect of the invention, the composition is produced by a method comprising:
(i) producing a lipid-in-water nano-emulsion comprising the lipid droplets, the active substance, and the inulin, wherein the inulin is dissolved in the aqueous phase of the nano-emulsion; and
(ii) spray-drying the nano-emulsion.

In some embodiments of the eleventh aspect of the invention, the nano-emulsion is produced by homogenizing a mixture comprising the lipid droplets and the active substance, and adding the inulin in aqueous form to the mixture.

In some embodiments of the eleventh aspect of the invention, the microparticles have an average diameter of <15 μm.

In a twelfth aspect, the present invention provides a method for treating or preventing a disease or disorder in a subject, the method comprising administering the composition of the eleventh aspect of the invention to the subject.

In a thirteenth aspect, the present invention provides a method for treating or preventing a microbial infection is a subject, the method comprising administering the composition of the eleventh aspect of the invention to the subject, wherein the pharmaceutical agent is an antimicrobial agent.

In some embodiments, the microbial infection is a bacterial infection. In some embodiments, the bacterial infection is due to Staphylococcus aureus. In some embodiments, the bacterial infection forms part of a biofilm.

In a fourteenth aspect, the present invention provides a method for administering an active substance to a subject, wherein the method comprises administering to the subject a composition of the eleventh aspect of the invention.

In a fifteenth aspect, the present invention provides a method for producing a composition of the eleventh aspect of the invention, wherein the method comprises spray-drying a lipid-in-water nano-emulsion comprising the lipid droplets, the active substance, and the inulin.

In a sixteenth aspect, the present invention provides a dry composition comprising:
(i) inulin;
(ii) lipid droplets; and
(iii) lecithin,
wherein the lipid droplets are encapsulated within polymeric chains of the inulin, and wherein the inulin is not in a nano-particulate form.

In a seventeenth aspect, the present invention provides a method for improving the gastrointestinal health of a subject, the method comprising administering to the subject a dry composition comprising:
(i) inulin;
(ii) lipid droplets; and
(iii) lecithin,
wherein the lipid droplets are encapsulated within polymeric chains of the inulin, and wherein the inulin is not in a nano-particulate form.

In an eighteenth aspect, the present invention provides a method for treating or preventing a metabolic disorder or disease in a subject, the method comprising administering to the subject a dry composition comprising:
(i) inulin;
(ii) lipid droplets; and
(iii) lecithin,
wherein the lipid droplets are encapsulated within polymeric chains of the inulin, and wherein the inulin is not in a nano-particulate form.

In a nineteenth aspect, the present invention provides a dry composition comprising:
(i) inulin;
(ii) lipid droplets; and
(iii) a poorly water-soluble active substance,
wherein the lipid droplets are encapsulated within polymeric chains of the inulin, and wherein the inulin is not in a nano-particulate form.

In a twentieth aspect, the present invention provides a method for treating or preventing a disease or disorder in a subject, the method comprising administering to the subject a dry composition comprising:
(i) inulin;
(ii) lipid droplets; and
(iii) a poorly water-soluble active substance, wherein the lipid droplets are encapsulated within polymeric chains of the inulin, and wherein the inulin is not in a nano-particulate form.

In a twenty first aspect, the present invention provides a dry composition comprising:
(i) inulin;
(ii) lipid droplets; and
(iii) an antibiotic,
wherein the lipid droplets are encapsulated within polymeric chains of the inulin, and wherein the inulin is not in a nano-particulate form.

In a twenty second aspect, the present invention provides a method for treating or preventing a bacterial infection in a subject, the method comprising administering to the subject a dry composition comprising:
(i) inulin;
(ii) lipid droplets; and
(iii) an antibiotic,
wherein the lipid droplets are encapsulated within polymeric chains of the inulin, and wherein the inulin is not in a nano-particulate form.

### BRIEF DESCRIPTION OF THE FIGURES

For a further understanding of the aspects and advantages of the present invention, reference should be made to the following detailed description, taken in conjunction with the accompanying figures which illustrate certain embodiments of the present invention.

FIG. 1—Schematic representation of the two-step fabrication process for Rif-ILH microparticles used in Example 1: (A) an inulin solution and (B) a rifampicin-loaded homogenized lipid emulsion (C) are combined, and (D) ultimately spray dried to form Rif-ILH microparticles.

FIG. 2—Scanning electron microscope (SEM) images of dry powder aggregates of raw inulin (A and B) and rifampicin-loaded inulin-lipid hybrid (Rif-ILH) microparticles (1:1 lipid:inulin ratio) (C and D) of Example 1.

FIG. 3—Rifampicin release kinetics from Rif-Lipid (blue) and Rif-ILH microparticles (pink) in (A) neutral media (PBS, pH 7.4, 37° C.) and (B) artificial lysosomal fluid (ALF; pH 4.5, 37° C.). Data is represented as mean±SD (n=3). (C) Schematic representation illustrating the release behaviour of rifampicin from both Rif-Lipid and Rif-ILH microparticles in (C) neutral media (pH 7.4), and (D) acidic media (pH 4.5).

FIG. 4—Cellular viability of inulin-lipid hybrid (ILH) microparticles (pink bars) and lipid micro-droplets (blue bars) in RAW 264.7 cells determined via MTT assay (mean±SD, n=3).

FIG. 5—Cellular uptake of lipid micro-droplets (blue bars) and ILH microparticles (pink bars) loaded with Nile red in RAW 264.7 cells after (A) 1 h and (B) 4 h incubation. No significant difference was observed between cells treated with Nile red (yellow bars) compared to the control group (no treatment; green bars).

FIG. 6—Confocal microscopy images for the uptake of Nile red formulations by RAW 264.7 macrophages, after 1 h incubation with: (A) Nile red solution, (B) Nile red-loaded lipid microparticles, and (C) Nile-red loaded ILH microparticles. Magnified regions of (D) Nile red-loaded lipid micro-droplets, and (E) Nile red-loaded ILH microparticles. All formulations were dosed at an equivalent dye concentration of 50 μg/mL. Nuclei were stained with DAPI dye (blue region); the cellular cytoskeleton was stained with Alexa-488 (green region); and the particles were stained with Nile red (red region).

FIG. 7—Internal rifampicin concentration within macrophages when incubated with a rifampicin solution (orange bars), Rif-Lipid (blue bars) and Rif-ILH microparticles (pink bars), when dosed at a rifampicin concentration 50 μg/mL after (A) 1 h and (B) 4 h incubation time (mean±SD, n=3).

FIG. 8—The intracellular presence of SCV S. aureus as observed using confocal microscopy. The nuclei and the cell membrane of macrophages were shown as purple and green, respectively. The localised aggregation of dye associated with the bacterial cell wall within the cells (as indicated by white arrows), demonstrate the presence of SCV S. aureus within cells.

FIG. 9—Efficacy of rifampicin-loaded formulations on the quantitative reduction of intracellular SCV S. aureus.

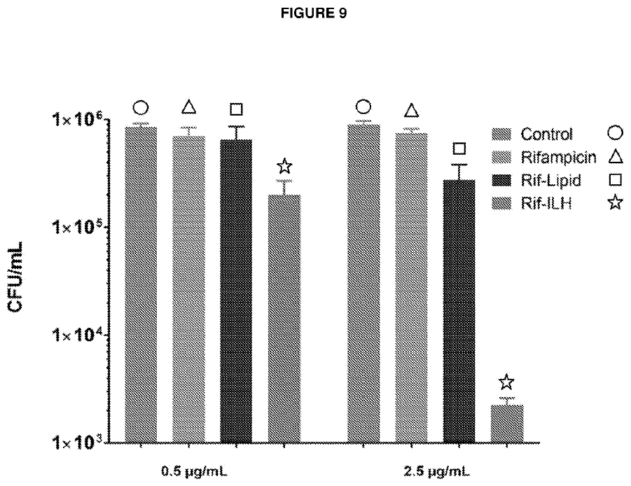

FIG. 10—Scanning electron micrograph (SEM) image of spray dried Inulin-lipid hybrid (ILH) microparticles. The scale bars represent 3 μm.

FIG. 11—A graph showing the biofilm biomass of S. aureus quantified by crystal violet staining at different concentration of rifampicin (1-16 μg/mL). Data represented as mean±standard deviation, n=6.

FIG. 12—A graph showing the biofilm biomass of S. aureus quantified by crystal violet staining following treatment with DMSO, rifampicin dissolved in DMSO (DMSO-Rif), Nanoemulsion (NanoE), rifampicin-solubilised nanoemulsion (NanoE-Rif), Inulin-lipid hybrid (ILH) and rifampicin-loaded ILH (ILH-Rif) for 24 h. Data represented as mean±standard deviation, n=6, ANOVA P>0.99.

FIG. 13—Scanning electron micrograph (SEM) images of a) raw inulin, and b) spray dried ILH with an inulin:lipid ratio of 50:50.

FIG. 14—Graph showing drug crystallinity of ILH formulations and crystalline drug measured using differential scanning calorimetry DSC.

FIG. 15—Graph showing in vitro dissolution evaluation of the percentage of fenofibrate dissolved over 90 min of ILH formulations compared to crystalline drug, commercial drug and emulsion [mean±SD (n=3)].

FIG. 16—Graph showing the results of in vitro lipolysis evaluation of the percentage of fenofibrate solubilised during digestion of ILH formulations, crystalline drug, commercial drug, liquid emulsion, and physical mix [mean±SD (n=3)].

FIG. 17—Graph showing the number of pmol of fatty acids released during 60 min intestinal phase (pH 6.5) corresponding to the amount of NaOH released from the digestion of lipids in the formulations [mean±SD (n=3)].

FIG. 18—Graph summarizing cumulative rodent weight gain normalized with respect to the initial body weight for the control group (PBS; grey circles), inulin (red squares), and ILH microparticles (green triangles). Inset:inulin and ILH microparticles reveal statistical significance (p<0.001) in AUC_{body weight %} after 21 days, when compared to the control group. Each value represents mean±SD, n=6.

FIG. 19—Graphs showing the impact of administering inulin and ILH microparticles on key metabolic biomarkers related to obesity: (A) triglycerides, (B) high density lipoprotein (HDL), (C) glucose, and (D) HbA1c. Each value represents mean±SD, n=6.

FIG. 20—Graph showing the relative abundance of health-promoting bacteria (Blautia, blue bars) and pathogenic bacteria (Proteobacteria, purple bars) in fecal and cecal contents of Sprague-Dawley rats, following daily dosing of ILH microparticles for 21 days, compared to a control group.

### DETAILED DESCRIPTION OF THE INVENTION

As set out above, the present invention is predicated, in part, on the identification of a dry composition which provides efficient delivery of active substances (such as poorly water-soluble drugs) to subjects in need thereof. The composition encompassed by the present invention also enables delivery of antimicrobial agents to microbial cells and biofilms. Furthermore, when administered alone to a subject (i.e. in the absence of an active substance), the dry composition encompassed by the present invention provides a health benefit, including a gastrointestinal health benefit, to the subject.

Accordingly, certain disclosed embodiments provide compositions and methods that have one or more advantages. For example, some of the advantages of some embodiments disclosed herein include one or more of the following: a novel composition with improved and efficient active substance delivery ability; a novel composition for improving health of a subject, including gastrointestinal health; a novel composition for the delivery of poorly water-soluble drugs to a subject; a novel composition for the delivery of active substances to microbial cells and biofilms; methods for improving the health of a subject, including gastrointestinal health; methods for treating or preventing a metabolic disorder or disease in a subject; methods for treating or preventing microbial infection in a subject; or the provision of a commercial alternative to existing compositions and methods. Other advantages of some embodiments of the present disclosure are provided herein.

The ability to efficiently deliver active substances to subjects for preventative or therapeutic applications has, and continues to be, a challenge for formulation chemists. To this end, nanotechnology research has paved the way toward more adaptive systems for drug delivery. Lipid-based compositions (such as liposomes and solid lipid nanoparticles) which encapsulate active substances have been widely investigated and some compositions of this type have been applied in particular drug delivery applications. However, wide-spread adoption of such delivery systems has been limited due to instability, and drug loading and release issues of the encapsulated agents. Nanocarriers such as polymeric nanoparticles have also been developed which provide higher levels of stability and improved drug release characteristics; however, the biocompatibility of many polymeric nanoparticles is not as high as lipid systems.

Hybrid systems, which combine the benefits of lipid-based technology with those of polymeric nanoparticles, have also been developed. These “hybrid” polymer-lipid nanocomposites have included mixing anionic poly(D,L-lactide-co-glycolide) (PLGA) nanoparticles with cationic liposomes; the use of phospholipids as emulsifiers in nanoparticle synthesis to form lipid-coated polymer nanoparticles such as PLGA-lipid hybrids; and encapsulating lipid within a three-dimensional porous silica matrix to form silica-lipid hybrid (SLH) microparticles.

More recent hybrid systems have included combining polymeric nanoparticles (such as PLGA nanoparticles) with lipid droplets to form dry polymer (nanoparticle)-lipid hybrid (PLH) microparticles. Such systems are formed by spray drying a PLGA-nanoparticle stabilised lipid emulsion to create hybrid microparticles where lipid is encapsulated within a three-dimensional polymeric matrix. To date, the efficacy of such systems remains largely untested in the clinic.

With the aim of developing an improved carrier system, including for the delivery of active substances to subjects, and for being adaptable to scale-up for mass manufacturing, the present Applicant has built on existing hybrid systems to develop a novel composition which does not rely on the use of polymeric nanoparticles. Instead, the carrier system developed by the present Applicant relies on the combination of lipid droplets and a polysaccharide (which is not in a nano-particulate form), in which the lipid droplets are encapsulated within polymeric chains of the polysaccharide. The formation of a dry powder comprising these components provides unexpected and advantageous delivery properties to the composition. Furthermore, when formulated with an active substance, the composition eliminates the need for biolabile conjugates between the polymeric chains of the polysaccharide and the active substance by encapsulating the active substance within the lipid nano-droplet phase.

Accordingly, in a first aspect the present invention provides a dry composition comprising:
(i) a polysaccharide; and
(ii) lipid droplets,
wherein the lipid droplets are encapsulated within polymeric chains of the polysaccharide, and wherein the polysaccharide is not in a nano-particulate form.

Reference herein to a “dry” composition indicates that the composition may be in the form of a dry substance comprising loose, aggregated, and/or partially aggregated microparticles. A “dry composition” as used herein is to be understood to mean that the composition comprises less than about 10 weight percent (wt %) of water, and more preferably less than about 5 wt % of water. Accordingly, the composition will typically be in the form of a free-flowing powder with no requirement for an anticaking agent such as calcium carbonate or powdered cellulose.

The size of the microparticles comprising a composition of the present invention will typically have an average diameter of less than 15 μm. For example, the average diameter of the microparticles may be in the range of about 1 μm to about 15 μm. In some embodiments, the average diameter of the microparticles may be in the range of about 1 μm to about 10 μm, about 1 μm to about 5 μm, about 2 μm to about 10 μm, about 2 μm to about 5 μm, about 3 μm to about 10 μm, or about 3 μm to about 5 μm. The microparticles typically have a spherical shape and smooth surface morphology as viewed by scanning electron microscopy. The microparticles in the composition may be present in the form of larger aggregates which may have an average maximum dimension in the range of 20 μm to 100 μm.

A “polysaccharide” for use in a composition of the present invention refers to a long chain polymeric carbohydrate composed of monosaccharide units bound together by glycosidic linkages. The monosaccharide units may be heterogeneous (forming a heteropolysaccharide) or may be homogeneous in nature (forming a homopolysaccharide). Examples of monosaccharides include, but are not limited to, glucose, fructose, mannose, galactose, xylose, and glyceraldehyde. The number of monosaccharide units in a polysaccharide, which is termed its degree of polymerization (DP), can vary depending on the polysaccharide type. The level of DP may be less than 100 but is typically more than 10. For some polysaccharides such as cellulose and starch, the DP is over 7,000 and 100,000, respectively.

The polysaccharide may be naturally occurring or synthetically made. Examples of a naturally occurring polysaccharide includes a dietary polysaccharide which can be found in daily food ingredients and in natural products such as plants. Starch, cellulose, pectin, chitosan, chitin, alginate, xyloglucan, β-glucan, xanthan gum, arabinoxylan, carrageenan, inulin, glucomannan, agar and plant gums are examples of common dietary polysaccharides. Cellulose consists a linear chain of several hundred to many thousand β(1→4) linked D-glucose units. Starch (amylose) has α(1→4) glycosidic bonds connecting α-D-glucose units, while starch (amylpectin) consists of glucose units linked in a linear manner with α(1→4) glycosidic bonds and α(1→6) bonds on branches which occur every 24 to 30 glucose units. Inulin possesses β(2,1) bonds that connect terminating glucosyl moieties and repetitive fructosyl moieties. Glucomannan consists of β(1→4) linked D-mannose and D-glucose units in a ratio of 1.6:1. β-Glucan is composed of β-D-glucose moieties that form a linear backbone with 1-3 β-glycosidic bonds. Pectins preferentially form linear chains of α-(1-4)-linked D-galacturonic acid, however other saccharide residues such as D-xylose, D-apiose, rhamnose, D-galactose, and L-arabinose may also present in the branching and linear chains inserted as random sequences. Xanthan has the monosaccharides β-D-glucose, α-D-mannose and α-D-glucoronic acid that are found in a ratio of 2:2:1 and linked with β-(1->4) glycosidic linkage. Arabinoxylan consists of long chains of 1,4-linked xylose units. Arabic gum contains a complex chemical structure comprising contiguous hydroxyprolines that are attached to oligo-α-1,3-L-arabinofurans and non-contiguous hydroxyprolines attached to galactose residues of oligo-arabinogalactans. Xyloglucan contains a core chain of β1→4-linked glucose moieties, which are substituted with 1-6 linked xylose sidechains. Synthetic polysaccharides can include those which have the same structure as their naturally occurring counterparts except that they have been artificially created. Synthetic polysaccharides can also encompass modifications which have been made to their naturally occurring counterparts, such as the addition of active chemical entities (for example in the case of methylated cellulose). Such synthetic polysaccharides encompassing modifications may therefore be considered analogues of naturally occurring polysaccharides. Whether naturally occurring or synthetically made, the polysaccharide for use in the present invention is soluble in an aqueous solution.

The type of polysaccharide used in a composition of the present invention is not limited, provided that the polymeric chains of the polysaccharide can encapsulate the lipid droplets of the composition. By “encapsulate the lipid droplets” is meant that the lipid droplets are retained within the final dry form of the composition, for example following a spray drying process.

Furthermore, the polysaccharide is not in a nano-particulate form. That is, the polysaccharide remains in an aqueous form, rather than in the form of a nanoparticle, prior to a spray drying process. Ultimately, this means that during spray drying, the polysaccharide coats the lipid droplets so that they remain encapsulated within the core of the microparticles comprising the composition.

In some embodiments, the polysaccharide is selected from the group consisting of inulin, cellulose and glucomannan. In some embodiments, the polysaccharide is inulin. Inulin is industrially most often extracted from chicory and is a class of dietary fibre known as fructan. However, inulin is found in more than 36,000 species of plants including agave, wheat, onion, dahlia, banana, garlic and asparagus. The extraction process for inulin from chicory plants includes slicing and washing chicory roots, soaking in a solvent, and subsequent isolation, purification and spray drying of the inulin component (see for example Belval H, 1927, Industrie de l'inuline et du lévulose. Dix Ans d'Efforts Scientifiques, Industriels et Coloniaux 1914-1924: 1068-1069 Chimie et Industrie Paris, France). Inulin may also be synthesized from sucrose. Inulin has the CAS Registry number 9005-80-5, cellulose has the CAS Registry number 9004-34-6, and glucomannan has the CAS Registry number 11078-31-2. Polysaccharides for use in the present invention can be purchased from commercial sources such as Sigma-Aldrich (Castle Hill, Australia).

The polysaccharide may be present in a composition of the present invention in an amount ranging from about 10 wt % to about 95 wt %, wherein the wt % amount is based on the total weight of the composition. For example, the polysaccharide may comprise about 25 wt % to about 90 wt %, about 50 wt % to about 70 wt %, about 15 wt %, about 20 wt %, about 25 wt %, about 30 wt %, about 35 wt %, about 40 wt %, about 45 wt %, about 50 wt %, about 55 wt %, about 60 wt %, about 65 wt %, about 70 wt %, about 75 wt %, about 80 wt %, about 85 wt %, or about 90 wt %, of the composition. Other ranges and values are contemplated.

The lipid which forms the lipid droplet component of a composition of the present invention may be natural or synthetic. Regardless, the lipid may be composed of mono-, di- and/or tri-substituted glycerols. As would be understood by a person skilled in the art, the lipid can be formed through the esterification of fatty acids with glycerol. For example, lipid is formed by linking glycerol to a C6 to C22 fatty acid acyl group. The acyl group may be branched or unbranched, saturated or unsaturated. In some embodiments, the acyl group is unbranched and saturated. In some embodiments, the acyl group may be derived from a saturated fatty acid, e.g., caprylic acid, capric acid, lauric acid, myristic acid, palmitic acid, stearic acid, arachidic acid, or behenic acid.

In some embodiments, the lipid may only comprise medium chain length fatty acids, i.e. fatty acids with aliphatic tails of 6 to 12 carbons. In this instance, the acyl group may be derived from, for example, caprylic acid (C8), capric acid (C10), and/or lauric acid (C12). However, it should be understood that lipid comprising long chain length fatty acids may also be used.

Methods for making a lipid for use in a composition of the present invention would be known in the art. For example, a lipid can be prepared through the glycerolysis of select fats and oils, or can be prepared by esterification of glycerin with specific fatty acids. Alternatively, a lipid may be obtained from coconut oil, or palm oil, or palm kernel oil by fractionated distillation followed by esterification with glycerol. Alternatively, the lipid may be purchased from commercial sources such as Abitec Pty Ltd (NSW, Australia) or Cremer Oleo GmbH & Co. KG (Hamburg, Germany). In this regard, in some embodiments the lipid may be selected from the group consisting of Capmul MCM, Capmul MCM C8, Capmul PG-8, Capmul MCM C10, Imwitor 742 and Imwitor 988.

The lipid may be present in a composition of the present invention in an amount ranging from about 10 wt % to about 95 wt %, wherein the wt % amount is based on the total weight of the composition. For example, the lipid may comprise about 30 wt % to about 50 wt %, about 15 wt %, about 20 wt %, about 25 wt %, about 30 wt %, about 35 wt %, about 40 wt %, about 45 wt %, about 50 wt %, about 55 wt %, about 60 wt %, about 65 wt %, about 70 wt %, about 75 wt %, about 80 wt %, about 85 wt %, or about 90 wt %, of the composition. Other ranges and values are contemplated.

The ratio of polysaccharide to lipid in a composition of the present invention may be in a range from about 10:90 to about 90:10. For example, the ratio may be about 20:80, about 30:70, about 40:60, about 50:50, about 80:20, about 70:30, or about 60:40, of polysaccharide to lipid in the composition. In some embodiments, the ratio of polysaccharide to lipid in a composition of the present invention may be in a range from about 50:50 to about 75:25. Other ratios within these ranges are contemplated.

A composition according to the first aspect and other aspects of the invention may comprise an active substance. The active substance may be any substance that requires delivery to a subject, a surface or natural matter. For example, an “active substance” as referred to herein may be a nutraceutical substance, a cosmetic substance, a pesticide compound, an agrochemical, a food stuff, or a pharmaceutical agent. It is to be understood that the active substance is not limited to the above examples.

In some embodiments of the first aspect and other aspects of the invention, the pharmaceutical agent is a drug or other biologically active molecule (for example a protein such as an antibody or antibody fragment, a peptide such as an antigenic peptide for the purposes of vaccination, or a nucleic acid molecule such as an oligonucleotide, an aptamer, a small interfering RNA, and the like).

A composition of the present invention is particularly suitable for the delivery of poorly water-soluble drugs to a subject. A poorly water-soluble drug would be understood by a person skilled in the art to be a lipophilic substance with a low aqueous dissolution. Poorly water-soluble drug candidates often emerge from contemporary drug discovery programs, and present formulators with considerable technical challenges. The absorption of such compounds when presented in the crystalline state to the gastrointestinal tract is typically dissolution rate-limited and therefore the oral bioavailability of the drug is limited.

Examples of poorly water-soluble drugs for use in the present invention include, but are not limited to: anti-inflammatory agents including celecoxib (4-[5-(4-methylphenyl)-3-(trifluoromethyl)pyrazol-1-yl]benzenesulfonamide), indomethacin (1-(4-chlorobenzoyl)-5-methoxy-2-methyl-1-H-indole-3-acetic acid, valdecoxib (4-(5-methyl-3-phenylisoxazol-4-yl) benzenesulfonamide), meloxicam ((8E)-8-[hydroxy-[(5-methyl-1,3-thiazol-2-yl)amino[methylidene)-9-methyl-10,10-dioxo-10λ⁶-thia-9-azabicyclo[4.4.0]deca-1,3,5-trien-7-one), rofecoxib (4-(4-methylsulfonylphenyl)-3-phenyl-5H-furan-2-one), diclofenac (2-(2-(2,6-dichlorophenylamino)phenyl)acetic acid), naproxen ((+)-(S)-2-(6-methoxynaphthalen-2-yl)propanoic acid) and combinations thereof; anti-cancer agents such as paclitaxel, 7-Ethyl-10-hydroxy-camptothecin (SN-38), etoposide, taxotere, docetaxel, temozolomide and combinations thereof; anti-emetic agents such, as cinnarizine ((E)-1-(Diphenylmethyl)-4-(3-phenylprop-2-enyl)piperazine); vitamins and derivatives thereof including vitamin B, vitamin D, retinol (vitamin A) and retinoic acid; antibiotic agents including tetracycline, rifampicin, clarithromycin, erythromycin and combinations thereof; anti-psychotic drugs including lurasidone, ziprasidone, and aripriprazole; and cholesterol-lowering drugs such as a statin or fenofibrate.

In some embodiments, a pharmaceutical agent for use in a composition of the present invention is selected from the group consisting of an antimicrobial agent, an anti-inflammatory agent, an antihistamine, a cholesterol-lowering drug, or a psychotropic drug. These agents may or may not be poorly water-soluble.

The antimicrobial agent may be one or more of an antibiotic, an antimicrobial peptide, and an antifungal agent. Antibiotics for use in a composition of the present invention may be selected from the group consisting of a protein synthesis inhibitor, a cell wall synthesis inhibitor including beta-lactam antibiotics, beta-lactamase inhibitors and peptidoglycan synthesis inhibitors, a lipopeptide including daptomycin, a DNA synthesis inhibitor, a RNA synthesis inhibitor, a mycolic acid synthesis inhibitor including isoniazid, a mechanosensitive channel of large conductance (MscL), and a folic acid synthesis inhibitor, or a combination of the aforementioned antibiotics. Antibiotics for use in the present invention can be purchased from relevant commercial suppliers such as Sigma-Aldrich (Castle Hill, NSW, Australia), and methods for their use are known in the art, for example as described in “Therapeutic Guidelines—Antibiotic”, Version 15, 2014, published by eTG complete.

The antibiotic may be a cationic antibiotic. Cationic antibiotics contain a positive charge and are therefore hindered in their effectiveness against microbial biofilms or hindered in their entry into microbial cells.

Examples of antibiotics which are protein synthesis inhibitors include those which stop or slow the growth or proliferation of cells by inhibiting the processes that lead to protein production. Such protein synthesis inhibitors typically (but not always) act by disrupting the activity of the ribosome during translation of mRNA. Examples of antibiotics which are classed as protein synthesis inhibitors include, but are not limited to, tetracyclines (such as demeclocycline, doxycycline, minocycline, oxytetracycline and tetracycline, or derivatives thereof such as tigecycline), aminoglycosides (such as amikacin, gentamicin, kanamycin, neomycin, netilmicin, tobramycin, paromomycin, streptomycin and spectinomycin), phenicols (such as chloramphenicol or derivatives thereof such as thiamphenicol), macrolides (such as azithromycin, clarithromycin, dirithromycin, erythromycin, roxithromycin, troleandomycin, telithromycin and spiramycin), lincosamides (such as clindamycin and lincomycin), fusidic acid, puromycin, streptogramins (such as pristinamycin, dalfopristin and quinupristin), retapamulin, ethionamide, mupirocin, oxazolidinones (such as linezolid, posizolid, radezolid and torezolid) and telithromycin.

Examples of antibiotics which are cell wall synthesis inhibitors include, but are not limited to, carbapenems (such as ertapenem, doripenem, imipenem and meropenem), penicillins (such as amoxicillin, ampicillin, azlocillin, carbenicillin, cloxacillin, dicloxacillin, fluloxacillin, mezlocillin, methicillin, nafcillin, oxacillin, penicillin G, penicillin V, piperacillin, temocillin and ticarcillin), cephalosporins (such as cefadroxil, cefazolin, cefalotin, cephalexin, cefaclor, cefamandole, cefoxitin, cefprozil, cefuroxime, cefixime, cefdinir, cefditoren, cefoperazone, cefotaxime, cefpodoxime, ceftazidime, ceftibuten, ceftizoxime, ceftriaxone, cefepime, ceftaroline fosamil and ceftobiprole), monobactams (such as aztreonam), fosfomycin, polymyxin B, bacitracin, colistin, glycopeptides (such as teicoplanin, vancomycin, telavancin, dalbavancin and oritavancin), and beta-lactamase inhibitors (such as clavulanic acid, sulbactam, tazobactam, tebipenem, avibactam and relebactam).

Examples of antibiotics which are DNA synthesis inhibitors include, but are not limited to, quinolones or fluoroquinolones (such as ciprofloxacin, enoxacin, gatifloxacin, gemifloxacin, levofloxacin, lomefloxacin, moxifloxacin, nalidixic acid, norfloxacin, ofloxacin, trovafloxacin, grepafloxacin, sparfloxacin and temafloxacin), and metronizadole.

Examples of antibiotics which are RNA synthesis inhibitors include, but are not limited to, rifamycins such as rifampin (rifampicin) and rifapentine.

Mechanosensitive channels of large conductance (MscL) consists of pore-forming membrane proteins that are responsible for translating physical forces applied to cell membranes into electrophysiological activities. MscL have a relatively large conductance, 3 nS, making them permeable to ions, water, and small proteins when opened. Examples of antibiotics which are MscL can be found at http://www.tcdb.org/search/result.php?tc=1.A.22.3. One specific example is Ramizol, which belongs to the styrylbenzene class of antibiotics.

Examples of antibiotics which are folic acid synthesis inhibitors include, but are not limited to, sulfonamides (such as mafenide, sulfacetamide, sulfadiazine, silver sulfadiazine, sulfadimethoxine, sulfamethizole, sulfamethoxazole, sulfanilamide, sulfasalazine, sulfisoxazole, trimethoprim-sulfamethoxazole and sulfonamidochrysoidine) and pyrimethamine.

The antibiotics for use in a composition of the present invention may also be selected from the group consisting of geldanamycin, herbimycin, rifaximin, furazolidone, nitrofurantoin, clofazimine, dapsone, capreomycin, cycloserine, ethambutol, pyrazinamide, rifabutin, arsphenamine, platensimycin and tinidazole.

In some embodiments, the antibiotic for use in a composition of the present invention is an aminoglycoside. Aminoglycosides are natural or semisynthetic antibiotics derived from actinomycetes. They share a core structure of amino acid sugars connected via glycosidic linkages to a dibasic aminocyclitol, which is most commonly 2-deoxystreptamine. Aminoglycosides are broadly classified into four subclasses based on the identity of the aminocyclitol moiety: (1) no deoxystreptamine (but rather having a streptidine ring); (2) a mono-substituted deoxystreptamine ring; (3) a 4,5-di-substituted deoxystreptamine ring; or (4) a 4,6-di-substituted deoxystreptamine ring. The core structure is decorated with a variety of amino and hydroxyl substitutions that have a direct influence on the mechanisms of action and susceptibility to various aminoglycoside-modifying enzymes (AMEs) associated with each of the aminoglycosides.

The aminoglycosides primarily act by binding to the aminoacyl site of 16S ribosomal RNA within the 30S ribosomal subunit, leading to misreading of the genetic code and inhibition of translocation. The initial steps required for peptide synthesis, such as binding of mRNA and the association of the 50S ribosomal subunit, are uninterrupted, but elongation fails to occur due to disruption of the mechanisms for ensuring translational accuracy. The ensuing antimicrobial activity is usually bactericidal against susceptible aerobic gram-negative bacilli.

The most common clinical application (either alone or as part of combination therapy) of the aminoglycosides is for the treatment of serious infections caused by aerobic gram-negative bacilli. While less common, aminoglycosides (in combination with other agents) have also been used for the treatment of select gram-positive infections. In addition, certain aminoglycosides have demonstrated clinically relevant activity against protozoa, Neisseria gonorrhoeae, and mycobacterial infections.

In some embodiments, the antimicrobial agent for use in a composition of the present invention is an antimicrobial peptide. The term “antimicrobial peptide” as used herein refers to a peptide that can kill or inhibit growth of a microorganism. The antimicrobial peptide may be a naturally occurring peptide, or may be artificially produced. Naturally occurring antimicrobial peptides are evolutionarily conserved molecules found in organisms ranging from prokaryotes to humans. Antimicrobial peptides are also referred to as peptide antibiotics.

Antimicrobial peptides generally consist of between 10 and around 50 amino acid residues. These peptides frequently contain a distribution of basic amino acids and hydrophobic residues that align in three dimensions on opposing faces, therefore forming unique structures that are water soluble, positively charged and hydrophobic. Folded cationic antimicrobial peptides can be classified into groups based on their secondary structure, namely α-helical, β-sheet, and extended antimicrobial peptides. Amphipathic α-helical antimicrobial peptides include the frog magainin, and the human cathelicidin peptide LL37. These peptides exhibit little secondary structure in aqueous solution but adopt the amphipathic α-helical architecture when they enter a non-polar environment, such as the bacterial membrane. Other antimicrobial peptides, such as bactenecins and defensins, are characterized by two or more β-sheets that are stabilized by disulfide bonds. Lastly, the extended antimicrobial peptides are peptides that do not possess a specific structural motif but rather are defined by a high content of specific residues, such as histidine, arginine, glycine or tryptophan. For example, histatins from humans are rich in histidine residues, and indolicidin from bovine leukocytes has multiple tryptophan and arginine residues.

More than 2,500 antimicrobial peptides have been identified in single-celled organisms, plants, insects and animals, and a number of them have been used as therapeutic agents in humans. These include the clinical use of: bacitracin for pneumonia; boceprevir for hepatitis C; and dalbavancin, daptomycin, oritavancin, telavancin and vancomycin for bacterial infections.

In some embodiments, the antimicrobial agent for use in a composition of the present invention is an antifungal agent. An “antifungal” as used herein means a biocidal compound that can inhibit the growth of, or kill, fungi or fungal spores. In some embodiments, the antifungal may be selected from one or more of a polyene, an azole, an allylamine, and an echinocandin.

A polyene is a molecule with multiple conjugated double bonds. A polyene antifungal is a macrocyclic polyene with a heavily hydroxylated region on the ring opposite the conjugated system. This makes polyene antifungals amphiphilic. Polyene antimycotics bind with sterols in the fungal cell membrane, principally ergosterol. This changes the transition temperature of the cell membrane, thereby placing the membrane in a less fluid, more crystalline state. As a result, the contents of the fungal cell leak and result in cell death.

In some embodiments, the polyene antifungal is selected from one or more of amphotericin B, candicidin, filipin, hamycin, natamycin, nystatin and rimocidin.

An azole antifungal can inhibit the enzyme lanosterol 14 α-demethylase, which is necessary to convert lanosterol to ergosterol. Depletion of ergosterol in fungal membrane disrupts the structure and many functions of the membrane ultimately leading to inhibition of fungal growth.

In some embodiments, the azole antifungal is selected from an imidazole, a triazole, and/or a thiazole. For example, the imidazole may be selected from bifonazole, butoconazole, clotrimazole, econazole, fenticonazole, isoconazole, ketoconazole, luliconazole, miconazole, omoconazole, oxiconazole, sertaconazole, sulconazole and tioconazole. The triazole may be selected from albaconazole, efinaconazole, epoxiconazole, fluconazole, isavuconazole, itraconazole, posaconazole, propiconazole, ravuconazole, terconazole and voriconazole. The thiazole may include abafungin.

An allylamine can inhibit squalene epoxidase, which is another enzyme required for ergosterol synthesis in the fungal membrane. In some embodiments, the allylamine antifungal may be selected from amorolfin, butenafine, naftifine, and terbinafine.

An echinocandin inhibits the synthesis of glucan in the cell wall via the enzyme 1,3-Beta-glucan synthase. In some embodiments, the echinocandin antifungal may be selected from anidulafungin, caspofungin and micafungin.

The antifungal for use in a composition of the present invention may also be selected from the group consisting of an aurone, benzoic acid, ciclopirox, flucytosine, griseofulvin, haloprogin, tolnaftate, undecylenic acid, crystal violet and Balsam of Peru.

Examples of fungal infections for which a composition of the present invention may be used include infections associated with a fungal species such as Aspergillus, Alternaria, Aureobasidium, Candida, Cladosporium, Cryptococcus, Curvularia, Coniophora, Diplodia, Epidermophyton, Engodontium, Fusarium, Gliocladium, Gloeophylium, Humicola, Histoplasma, Lecythophora, Lentinus, Malassezia, Memnionella, Mucor, Oligoporus, Paecilomyces, Penicillium, Petriella, Paracoccidioides, Phanerochaete, Phoma, Pneumocystis, Poria, Pythium, Rhodotorula, Rhizopus, Schizophyllum, Sclerophoma, Scopulariopsis, Serpula, Sporobolomyces, Stachybotrys, Stemphylium, Trichosporon, Trichtophyton, Trichurus, and Ulocladium. Other types of fungi are contemplated.

In some embodiments, the antibiotic is selected from the group consisting of rifampicin, tobramycin and vancomycin.

In some embodiments, the cholesterol-lowering drug is a statin or fenofibrate. In some embodiments, the statin is simvastatin.

In some embodiments, the psychotropic drug is lurasidone.

The active substance may be present in a composition of the present invention in an amount ranging from about 0.5 wt % to about 25 wt %, wherein the wt % amount is based on the total weight of the composition. For example, the active substance may comprise about 1.0 wt %, about 2.0 wt %, about 3.0 wt %, about 4.0 wt %, about 5.0 wt %, about 6.0 wt %, about 7.0 wt %, about 8.0 wt %, about 9.0 wt %, about 10 wt %, about 11 wt %, about 12 wt %, about 13 wt %, about 14 wt %, about 15 wt %, about 16 wt %, about 17 wt %, about 18 wt %, about 19 wt %, about 20 wt %, about 21 wt %, about 22 wt %, about 23 wt %, about 24 wt %, or about 25 wt %, of the composition. Other ranges and values are contemplated.

In some embodiments of the first and other aspects of the invention, the composition further comprises an excipient or stabilizer. These may be included during preparation of the components of the composition and/or when formulating the composition for delivery, and accordingly encompass pharmaceutically acceptable excipients or stabilizers. Suitable excipients or stabilizers would be known to a person skilled in the art, and for example can include lecithin, sodium dodecyl sulphate (SDS), polyoxyethylene-polyoxypropylene block copolymer surfactant (Pluronic® F-68; Sigma-Aldrich Co. LLC, St Louis, MO, United States of America), Pluronic® F-127 surfactant (Sigma-Aldrich Co. LLC), poloxamine, polyethylene glycol (PEG), polylactic acid (PLA), polyethylene glycol sorbitan monooleate (Tween® 80; Sigma-Aldrich Co. LLC), Vitamin E TP-GS (d-alpha tocopheryl polyethylene glycol 1000 succinate; Antares Health Products Inc., Batavia, IL, United States of America), polyvinyl alcohol (PVA) and didodecyldimethyl ammonium bromide (DMAB).

In some embodiments, the stabilizer is lecithin. Lecithin comprises a mixture of glycerophospholipids including phosphatidylcholine, phosphatidylethanolamine, phosphatidylinositol, phosphatidylserine, and phosphatidic acid rendering lecithin amphiphilic. Lecithin can be extracted chemically from sources including egg yolks, soybeans, milk, rapeseed, cottonseed and sunflower oil by the use of solvents such as hexane, ethanol, acetone or benzene. Lecithin can also be purchased commercially from sources such as Sigma Aldrich (Castle Hill, Australia).

Lecithin acts to prevent phase separation between the lipid and aqueous phases by adsorbing to the lipid/aqueous interface, thus stabilizing the lipid droplets. When preparing a composition of the present invention, lecithin can be first dissolved in the lipid and this mix is then added to water and stirred to form an emulsion. The emulsion is then ultrasonicating to create a lipid-in-water nano-emulsion in the form of lipid droplets.

The stabilizer may be present in a composition of the present invention in an amount ranging from about 0.05 wt % to about 10 wt %, wherein the wt % amount is based on the total weight of the composition. For example, the stabilizer may comprise about 0.1 wt %, about 0.15 wt %, about 0.2 wt %, about 0.25 wt %, about 0.3 wt %, about 0.35 wt %, about 0.4 wt %, about 0.45 wt %, about 0.5 wt %, about 1.0 wt %, about 1.5 wt %, about 2.0 wt %, about 2.5 wt %, about 3.0 wt %, about 3.5 wt %, about 4.0 wt %, about 4.5 wt %, about 5.0 wt %, about 5.5 wt %, about 6.0 wt %, about 6.5 wt %, about 7.0 wt %, about 7.5 wt %, about 8.0 wt %, about 8.5 wt %, about 9.0 wt %, about 9.5 wt %, or about 10 wt %, of the composition. Other ranges and values are contemplated.

A composition of the present invention can be formulated for administration to a subject or object to be treated. This means that the composition can take a number of physical forms depending on the nature of the use of the composition and required mode of administration. In this regard, one route of administration may be suitable for oral administration such as a tablet, pill, capsule, or may be in another dosage form useful for systemic administration of agents. For systemic administration, the composition may be in the form of an injectable solution. Another route of administration may include topical administration and therefore the composition may be in the form of a liquid, gel, suspension, paste, lotion, cream, solid, semi-solid, powder, and the like. The composition may also be in the form of an aerosol, nebulizer or dry powder for inhalation delivery. Other forms of administration may include delivery by way of a scaffold, such as a biomaterial scaffold including a scaffold produced from collagen, hydroxyapatite, β-tricalcium phosphate or a combination thereof. Other routes of administration are contemplated.

A composition of the present invention may be administered alone or may be delivered in the form of a suitable pharmaceutical composition, for example in a mixture with other therapeutic substances and/or other substances that enhance, stabilise or maintain the activity of the components of the composition. In some embodiments, an administration vehicle (e.g., liquid, gel, paste, powder, cream, pill, tablet, capsule, injectable solution, aerosol, etc) would contain the composition and/or additional substance(s). In this regard, the pharmaceutical composition may also include the use of one or more pharmaceutically acceptable carriers or additives, including pharmaceutically acceptable salts, amino acids, polypeptides, polymers, solvents, buffers, excipients and bulking agents, taking into consideration the particular physical and chemical characteristics of the composition to be administered.

In some embodiments, the carrier may be chosen based on various considerations including the route of administration, the active substance being delivered and the time course of delivery of the composition. The term “pharmaceutically acceptable carrier” refers to a substantially inert solid, semi-solid or liquid filler, diluent, excipient, encapsulating material or formulation auxiliary of any type. An example of a pharmaceutically acceptable carrier is physiological saline. Other physiologically acceptable carriers and their formulations are known in the art. Some examples of materials which can serve as pharmaceutically acceptable carriers include sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols such as propylene glycol; esters such as ethyl oleate and ethyl laurate; agar; detergents such as TWEEN 80; buffering agents such as magnesium hydroxide and aluminium hydroxide; alginic acid; pyrogen-free water; isotonic saline; Ringer's solution; ethyl alcohol; and phosphate buffer solutions, as well as other non-toxic compatible lubricants such as sodium lauryl sulfate and magnesium stearate, as well as colouring agents, releasing agents, coating agents, sweetening, flavouring and perfuming agents, preservatives and antioxidants can also be present.

The preparation of such pharmaceutical compositions is known in the art, for example as described in Remington's Pharmaceutical Sciences, 18th ed., 1990, Mack Publishing Co., Easton, Pa. and U.S. Pharmacopeia: National Formulary, 1984, Mack Publishing Company, Easton, Pa, which are incorporated herein by reference in their entirety.

A composition of the present invention provides particular advantages for oral delivery of active substances. Therefore, the composition is preferably formulated for oral delivery such as in the form of a tablet, pill, capsule, caplet, liquid emulsion, suspension or elixir, and the like. Oral medication is the most common form of drug administration because of advantages such as convenience of administration via the oral route, patient preference, cost-effectiveness, and ease of large-scale manufacturing of oral dosage forms. The compliance of patients to oral formulations is generally higher than that to other parenteral routes such as intravenous, subcutaneous, and intramuscular injections, as well as to inhalation medications. Furthermore, orally administered drugs can be targeted to particular regions within the gastrointestinal (GI) tract for localized treatment of pathological conditions such as stomach and colorectal cancers, infections, inflammations, bowel diseases, gastro-duodenal ulcers, and gastroesophageal reflux disorders.

Although oral administration is the preferred administration route of the composition of the present invention, the composition may also be formulated for other modes of administration. For example, the composition of the present invention may be formulated for parenteral administration. The term “parenteral” as used herein includes subcutaneous, intravenous, intramuscular, intraperitoneal, intrathecal, intraventricular, intrasternal, and intracranial injection or infusion techniques.

When administered parenterally, the composition will normally be in a unit dosage, sterile injectable, form (solution, suspension or emulsion) which is preferably isotonic with the blood of the recipient with a pharmaceutically acceptable carrier. Examples of such sterile injectable forms are sterile injectable aqueous or oleaginous suspensions. These suspensions may be formulated according to techniques known in the art using suitable dispersing or wetting agents and suspending agents. The sterile injectable forms may also be sterile injectable solutions or suspensions in non-toxic parenterally-acceptable diluents or solvents, for example, as solutions in 1,3-butanediol. Among the acceptable vehicles and solvents that may be employed are water, saline, Ringer's solution, dextrose solution, isotonic sodium chloride solution, and Hanks' solution. In addition, sterile, fixed oils are conventionally employed as solvents or suspending mediums. For this purpose, any bland fixed oil may be employed including synthetic mono- or di-glycerides, corn, cottonseed, peanut, and sesame oil. Fatty acids such as ethyl oleate, isopropyl myristate, and oleic acid and its glyceride derivatives, including olive oil and castor oil, especially in their polyoxyethylated versions, are useful in the preparation of injectables. These oil solutions or suspensions may also contain long-chain alcohol diluents or dispersants.

The carrier may contain minor amounts of additives, such as substances that enhance solubility, isotonicity, and chemical stability, for example anti-oxidants, buffers and preservatives.

In some embodiments, a composition of the present invention may be formulated for administration by direct introduction to the respiratory system (e.g. the lungs), such as by inhalation administration via aerosol, nebulizer, or dry powder, or by being instilled into the lung. In some embodiments, it may be desirable to administer the composition directly to the airways in the form of a dry powder, since high doses of medication can be delivered over shorter periods of time compared to that of a nebuliser without the associated risks of nebuliser induced damage to the antimicrobial composition ultrastructure and/or long-term stability issues from liquid storage. In order to develop an inhalable dry powder formulation, spray-drying, lyophilisation and milling techniques can be used to produce micron-sized powders.

In some embodiments, a composition of the present invention may be formulated for topical administration, e.g. transdermal administration. Transdermal administrations are understood to include all administrations across the surface of the body and the inner linings of bodily passages including epithelial and mucosal tissues. Such administrations may be carried out using the composition of the present invention as described herein, in the form of a liquid, gel, paste, lotion, cream, ointment, powder, foam, patch, suspension, solution, and a suppository (rectal and vaginal), or other suitable form.

A cream is a formulation that contains water and oil and is stabilized with an emulsifier. Lipophilic creams are called water-in-oil emulsions, and hydrophilic creams oil-in-water emulsions. The cream base for water-in-oil emulsions are normally absorption bases such as vaseline, ceresin or lanolin. The bases for oil-in-water emulsions are mono-, di-, and tri-glycerides of fatty acids or fatty alcohols with soaps, alkyl sulphates or alkyl polyglycol ethers as emulsifiers.

A lotion is an opaque, thin, non-greasy emulsion liquid dosage form for external application to the skin, which generally contains a water-based vehicle with greater than 50% of volatiles and sufficiently low viscosity that it may be delivered by pouring. Lotions are usually hydrophilic and contain greater than 50% of volatiles as measured by LOD (loss on drying). A lotion tends to evaporate rapidly with a cooling sensation when rubbed onto the skin.

A paste is an opaque or translucent, viscous, greasy emulsion or suspension semisolid dosage form for external application to the skin, which generally contains greater than 50% of hydrocarbon-based or a polyethylene glycol-based vehicle and less than 20% of volatiles. A paste contains a large proportion (20-50%) of dispersed solids in a fatty or aqueous vehicle.

An ointment is an opaque or translucent, viscous, greasy emulsion or suspension semisolid dosage form for external application to the skin, which generally contains greater than 50% of hydrocarbon-based or a polyethylene glycol-based vehicle and less than 20% of volatiles. An ointment is usually lipophilic and contains >50% of hydrocarbons or polyethylene glycols as the vehicle and <20% of volatiles as measured by LOD. An ointment tends not to evaporate or be absorbed when rubbed onto the skin.

A gel is usually a translucent, non-greasy emulsion or suspension semisolid dosage form for external application to the skin, which contains a gelling agent in quantities sufficient to impart a three-dimensional, cross-linked matrix. A gel is usually hydrophilic and contains sufficient quantities of a gelling agent such as starch, cellulose derivatives, carbomers, magnesium-aluminum silicates, xanthan gum, colloidal silica, aluminium or zinc soaps.

A composition of the present invention, when in a form for topical administration, may further include drying agents, anti-foaming agents, buffers, neutralizing agents, agents to adjust pH, colouring agents and decolouring agents, emollients, emulsifying agents, emulsion stabilizers and viscosity builders, humectants, odorants, preservatives, antioxidants, and chemical stabilizers, solvents, and thickening, stiffening, and suspending agents, and a balance of water or solvent.

Transdermal administration may also be accomplished through the use of a transdermal patch containing the active components of the composition and a carrier that is inert to the active components, is non-toxic to the skin, and allows delivery of the agent for systemic absorption into the blood stream via the skin. The carrier may take any number of forms such as creams and ointments, pastes, gels, and occlusive devices. The creams and ointments may be viscous liquid or semisolid emulsions of either the oil-in-water or water-in-oil type. Pastes comprised of absorptive powders dispersed in petroleum or hydrophilic petroleum containing the active ingredient may also be suitable. A variety of occlusive devices may be used to release the active ingredient into the blood stream such as a semi-permeable membrane covering a reservoir containing the active ingredient with or without a carrier, or a matrix containing the active ingredient. Transdermal formulations are known in art and may be formulated by a skilled person.

As indicated above, in some embodiments a composition of the present invention may be formulated for administration by way of a suppository. Suppository formulations may be made from traditional materials, including cocoa butter, with or without the addition of waxes to alter the suppository's melting point, and glycerin. Water soluble suppository bases, such as polyethylene glycols of various molecular weights, may also be used.

A composition of the present invention may also be formulated using controlled release technology. For example, the composition may be administered as a sustained-release pharmaceutical. To further increase the sustained release effect, the composition may be formulated with additional components such as vegetable oil (for example soybean oil, sesame oil, camellia oil, castor oil, peanut oil, rape seed oil); middle fatty acid triglycerides; fatty acid esters such as ethyl oleate; glycerol monooleate; polysiloxane derivatives; alternatively, water-soluble high molecular weight compounds such as hyaluronic acid or salts thereof (weight average molecular weight: ca. 80,000 to 2,000,000), carboxymethylcellulose sodium (weight average molecular weight: ca. 20,000 to 400,000), hydroxypropylcellulose (viscosity in 2% aqueous solution: 3 to 4,000 cps), atherocollagen (weight average molecular weight: ca. 300,000), polyethylene glycol (weight average molecular weight: ca. 400 to 20,000), polyethylene oxide (weight average molecular weight: ca. 100,000 to 9,000,000), hydroxypropylmethylcellulose (viscosity in 1% aqueous solution: 4 to 100,000 cSt), methylcellulose (viscosity in 2% aqueous solution: 15 to 8,000 cSt), polyvinyl alcohol (viscosity: 2 to 100 cSt), polyvinylpyrrolidone (weight average molecular weight: 25,000 to 1,200,000).

Alternatively, a composition of the present invention may be incorporated into a hydrophobic polymer matrix, scaffold or support (such as a biodegradable matrix or support), including for controlled release of the composition over a period of days. Methods for delivering agent(s) via scaffolds are known in the art. For example, a biomaterial scaffold including a scaffold produced from collagen, hydroxyapatite, β-tricalcium phosphate or a combination thereof may be used to deliver the agent. Methods for incorporating agent(s) into such substrates are known in the art.

A composition of the present invention may also be moulded into a solid implant, or externally applied patch, suitable for providing efficacious concentrations of the composition over a prolonged period of time without the need for frequent re-dosing. Such controlled release films are well known in the art. Other examples of polymers commonly employed for this purpose that may be used include nondegradable ethylene-vinyl acetate copolymer or degradable lactic acid-glycolic acid copolymers which may be used externally or internally. Certain hydrogels such as poly(hydroxyethylmethacrylate) or poly(vinylalcohol) also may be useful, but for shorter release cycles than the other polymer release systems, such as those mentioned above.

The carrier may also be a solid biodegradable polymer or mixture of biodegradable polymers with appropriate time release characteristics and release kinetics. The composition may then be moulded into a solid implant suitable for providing efficacious concentrations of the composition over a prolonged period of time without the need for frequent re-dosing. The composition can be incorporated into the biodegradable polymer or polymer mixture in any suitable manner known to one of skill in the art and may form a homogeneous matrix with the biodegradable polymer, or may be encapsulated in some way within the polymer, or may be moulded into a solid implant.

A composition according to the first aspect of the invention can be produced by a method comprising:
(i) producing a lipid-in-water nano-emulsion comprising the lipid droplets and the polysaccharide, wherein the polysaccharide is dissolved in the aqueous phase of the nano-emulsion; and
(ii) spray-drying the nano-emulsion.

For step (i), an emulsion is formed which contains the lipid. For example, the lipid may be added to an appropriate aqueous buffer or to water, to form the emulsion, which is then mixed so as to form lipid droplets in the emulsion. The mixing typically includes stirring the lipid in the aqueous buffer or water to form an emulsion. The emulsion is then homogenized. In an embodiment where water is used, a lipid-in-water nano-emulsion in the form of lipid droplets is created. The polysaccharide in an aqueous form (i.e. in solution) is then added to the lipid-in-water nano-emulsion and then mixed such that the polysaccharide is dissolved in the aqueous phase of the lipid-in-water nano-emulsion. The lipid droplets are encapsulated within polymeric chains of the polysaccharide as described above.

The amount of lipid contained in the lipid-in-water nano-emulsion is typically in the range of about 1% w/v to about 20% w/v. For example, the lipid is present in an amount of about 2% w/v, about 3% w/v, about 4% w/v, about 5% w/v, about 6% w/v, about 7% w/v, about 8% w/v, about 9% w/v, about 10% w/v, 11% w/v, about 12% w/v, about 13% w/v, about 14% w/v, about 15% w/v, about 16% w/v, about 17% w/v, about 18% w/v, or about 19% w/v, in the lipid-in-water nano-emulsion. Other ranges and values are contemplated. In some embodiments, the lipid is present in an amount of about 2% w/v in the lipid-in-water nano-emulsion.

The amount of polysaccharide contained in the lipid-in-water nano-emulsion is typically in the range of about 1% w/v to about 20% w/v. For example, the polysaccharide is present in an amount of about 2% w/v, about 3% w/v, about 4% w/v, about 5% w/v, about 6% w/v, about 7% w/v, about 8% w/v, about 9% w/v, about 10% w/v, 11% w/v, about 12% w/v, about 13% w/v, about 14% w/v, about 15% w/v, about 16% w/v, about 17% w/v, about 18% w/v, or about 19% w/v, in the lipid-in-water nano-emulsion. Other ranges and values are contemplated. In some embodiments, the polysaccharide is present in an amount of about 2% w/v in the lipid-in-water nano-emulsion.

In embodiments of the invention where the composition contains an active substance, the active substance may be contained within the aqueous phase of the composition containing the polysaccharide. For example, the active substance may be dissolved in the aqueous phase along with the polysaccharide. In this regard, step (i) of the method described above may first involve adding the lipid and the active substance to an appropriate buffer or to water, to form an aqueous solution prior to mixing and homogenization. In an embodiment where water is used, a lipid-in-water nano-emulsion is created. The polysaccharide in an aqueous form is then added to the lipid-in-water nano-emulsion and mixed to produce a lipid-in-water nano-emulsion comprising the lipid droplets, the active substance, and the polysaccharide.

The lipid-in-water nano-emulsion produced in step (i) may then be treated to produce a dry composition using techniques known in the art, for example by spray drying, freeze drying, or through the use of fluidised bed procedures. Accordingly, in one embodiment step (ii) referred to above comprises spray-drying the nano-emulsion produced in step (i). The spray drying will typically be conducted at a temperature greater than the glass transition temperature (tg) of the polysaccharide but can also be conducted at a temperature less than the tg of the polysaccharide. Other spray drying parameters, such as emulsion flow rate and air flow rate, are preferably set to provide a high level or optimal level of removal of residual moisture. Examples include an emulsion flow rate of <1 mL/min, such as in the range of about 0.25 to about 0.75 mL/min, and an air flow rate of <1 m³/min, such as in the range of about 0.25 to about 0.75 m³/min. When the polysaccharide is inulin, the emulsion flow rate is about 0.5 mL/min and the air flow rate is about 0.6 m³/min.

The spray drying step may be performed using any spray drying apparatus known to a person skilled in the art. For example, the Mini Spray Dryer B-290 from Buchi Labortechnik AG. When the polysaccharide is inulin, this spray dryer is used with an inlet temperature of 160° C., and outlet temperature of 90° C. and an aspirator setting of 100%.

In the absence of an active substance, a compound according to the first aspect of the present invention, when administered to a subject, has been shown to improve the health of the subject. In this regard, the Applicant has shown that treating rodents exposed to a high-fat diet with a composition of the invention (in which the polysaccharide is inulin) promotes metabolic health through a reduction in weight gain, and significantly promotes positive changes in key biomarkers linked with a heightened risk for developing metabolic syndrome/obesity, such as blood triglycerides, HDL, glucose and HbA1c levels. This suggests that formulation of a composition according to this aspect of the present invention provides a multifunctional mechanism of action, compared to inulin alone.

Accordingly, in a second aspect the present invention provides a method for improving the health of a subject, or for treating or preventing a disease or disorder in a subject, the method comprising administering the composition of the first aspect of the invention to the subject.

In some embodiments, the gastrointestinal health of the subject is improved. Poor gastrointestinal health is not uncommon and is associated with, contributes to, exacerbates, or causes, any number of conditions affecting the overall health and well-being of animals, particularly mammals. Conditions related to poor gastrointestinal health can be quite serious and require medical attention. They include, for example, Crohn's disease and irritable bowel disease, as well as other similar, chronic conditions. Other conditions related to poor gastrointestinal health that are less serious and can be essentially self-limiting include, for example, food-borne viruses and intestinal flu that often result in diarrhea, poor stool quality, or other symptoms of poor gastrointestinal health. Poor gastrointestinal health results from various causes. For example, intestinal bacterial overgrowth (IBO) occurs in people and companion animals such as dogs, cats, and horses. IBO may be caused by poor motility, food retention, or decreased gastric acidity. Further, animals must efficiently and properly digest food and absorb these dietary nutrients in order to maintain good health. However, poor gastrointestinal health can interfere with the ordinary digestion of food and adversely affect the health and well-being of animals. In some embodiments, the composition of the present invention improves the gastrointestinal health of the subject by increasing the relevant abundance of health-promoting bacteria in the gut and decreasing the relative abundance of pathogenic bacteria.

For example, in compositions of the present invention comprising inulin, the composition improves gastrointestinal health by increasing the relative abundance of health-promoting bacteria, such as Blautia sp, in the gut while reducing the relative abundance of pathogenic bacteria, such as Proteobacteria. The gastrointestinal health in a subject is improved when compared to a control subject which has not been administered the composition.

The gastrointestinal health of the subject may be improved by at least 1%, 5%, 10%, 20%, 30%, 40%, 50%, 60%, 70%, 80%, 90%, 100%, or greater, or by 1-fold, 2-fold, 3-fold, 4-fold, 5-fold, 6.0-fold, 7-fold, 8-fold, 9-fold, 10-fold, 15-fold, 20-fold, 25-fold, 30-fold, 35-fold, 40-fold, 45-fold, 50-fold, 60-fold, 70-fold, 80-fold, 90-fold, 100-fold, 125-fold, 150-fold, 175-fold, 200-fold, 225-fold, 250-fold, 275-fold, 300-fold, 400-fold, 500-fold, 1000-fold, 10,000-fold, 100,000-fold, or greater, when compared to the gastrointestinal health of a control subject which has not been administered the composition.

In some embodiments, a composition according to the first and other aspects of the invention may be used to treat or prevent a metabolic disease or disorder in the subject. A metabolic disease or disorder (also encompassing metabolic syndrome) in a subject is intended to mean a subject having one or more of the following risk factors and associated conditions: impaired blood glucose tolerance, elevated fasting blood glucose, abdominal obesity, high blood pressure, high serum triglyceride levels, low high-density lipoprotein (HDL) levels, HbA1c levels, insulin resistance, insulin insufficiency, hyperinsulinemia, Type 2 diabetes, and cardiovascular disease. Other risk factors and associated conditions of a metabolic disease or disorder are also encompassed by the present invention as would be known by a person skilled in the art.

In compositions of the present invention comprising inulin, the composition may promote a reduction of body weight, reduce blood triglyceride levels, increase HDL levels, reduce blood glucose levels, and reduce HbA1c levels compared to a control subject which has not been administered the composition. Given that these metabolic health factors are all linked with the gut microbiome and gut health, and without wishing to be bound by theory, compositions of the present invention comprising inulin may function by promoting a healthy gut microbiome, which in turn improves the metabolic health of the subject.

As indicated above, in some embodiments a composition of the present invention may comprise an active substance which is an antimicrobial agent. Accordingly, in a third aspect the present invention provides a method for treating or preventing a microbial infection in a subject, the method comprising administering a composition of the present invention, which comprises an antimicrobial agent, as described herein, to the subject. The composition may therefore be used to reduce the viability of a microorganism, enhance the activity of the antimicrobial agent in a subject, potentiate the activity of the antimicrobial agent in a subject, reduce the dose of the antimicrobial agent required to treat or prevent a microbial infection in a subject, increase the potency of the antimicrobial agent required to treat or prevent a microbial infection in a subject, or reduce viability of a microorganism resistant or tolerant to the antimicrobial agent. Other uses are contemplated.

The aforementioned treatment and prevention methods require administering to the subject an effective amount of the composition. The term “effective amount” as used herein is the quantity of the composition which, when administered to a subject, improves the prognosis and/or health state of the subject. The amount of composition to be administered to a subject will depend on one or more of the mode of administration of the composition, and the characteristics of the subject, such as general health, other diseases, age, sex, genotype, and body weight. When the composition comprises an antimicrobial agent, the amount of the composition administered will also depend on the level or amount of resistance or tolerance to the antimicrobial agent in the subject, and the type of infection being inhibited, prevented or treated. A person skilled in the art will be able to determine appropriate dosages depending on these and other factors. The effective amount of the composition to be used in the various embodiments of the present invention is not particularly limited.

In some embodiments of the third aspect of the invention, the antimicrobial agent is administered to the subject (as part of the composition) so as to expose the microorganism causing the infection in the subject to a concentration of the agent in the range from 0.1 μg/ml to 1,000 μg/ml, 1 μg/ml to 1,000 μg/ml, 10 μg/ml to 1,000 μg/ml, 100 μg/ml to 1,000 μg/ml, 500 μg/ml to 1,000 μg/ml, 0.1 μg/ml to 500 μg/ml, 1 μg/ml to 500 μg/ml, 10 μg/ml to 500 μg/ml, 100 μg/ml to 500 μg/ml, 0.1 μg/ml to 250 μg/ml, 1 μg/ml to 250 μg/ml, 10 μg/ml to 250 μg/ml, 100 μg/ml to 250 μg/ml, 0.1 μg/ml to 100 μg/ml, 1 μg/ml to 100 μg/ml, or 10 μg/ml to 100 μg/ml. Other ranges are contemplated with the ultimate amount dictated by the antimicrobial agent used.

In some embodiments of the third aspect of the invention, the antimicrobial agent is administered to the subject (as part of the composition) in an amount ranging from one of the following selected ranges: 1 μg/kg to 1000 mg/kg; 1 μg/kg to 100 mg/kg; 1 μg/kg to 10 mg/kg; 1 μg/kg to 1 mg/kg; 1 μg/kg to 100 μg/kg; 1 μg/kg to 10 μg/kg; 10 μg/kg to 1000 mg/kg; 10 μg/kg to 100 mg/kg; 10 μg/kg to 10 mg/kg; 10 μg/kg to 1 mg/kg; 10 μg/kg to 100 μg/kg; 100 μg/kg to 1000 mg/kg; 100 μg/kg to 100 mg/kg; 100 μg/kg to 10 mg/kg; 100 μg/kg to 1 mg/kg; 1 mg/kg to 1000 mg/kg; 1 mg/kg to 100 mg/kg; 1 mg/kg to 10 mg/kg; 10 mg/kg to 1000 mg/kg; 10 mg/kg to 100 mg/kg; and 100 mg/kg to 1000 mg/kg body weight of the subject. The dose and frequency of administration may be determined by one of skill in the art.

In some embodiments, the microorganism is a bacterium and therefore infection is due to a bacterium. In some embodiments, the bacterium comprises a Gram positive bacterium, a Gram negative bacterium, a Gram test non-responsive bacteria, an aerobic bacterium, or an anaerobic bacterium.

Examples of genera or species of bacterium include Abiotrophia, Achromobacter, Acidaminococcus, Acidovorax, Acinetobacter, Actinobacillus, Actinobaculum, Actinomadura, Actinomyces, Aerococcus, Aeromonas, Afipia, Agrobacterium, Alcaligenes, Alloiococcus, Alteromonas, Amycolata, Amycolatopsis, Anaerobospirillum, Anaerorhabdus, Arachnia, Arcanobacterium, Arcobacter, Arthrobacter, Atopobium, Aureobacterium, Bacteroides, Balneatrix, Bartonella, Bergeyella, Bifidobacterium, Bilophila Branhamella, Borrelia, Bordetella, Brachyspira, Brevibacillus, Brevibacterium, Brevundimonas, Brucella, Burkholderia, Buttiauxella, Butyrivibrio, Calymmatobacterium, Campylobacter, Capnocytophaga, Cardiobacterium, Catonella, Cedecea, Cellulomonas, Centipeda, Chlamydia, Chlamydophila, Chromobacterium, Chyseobacterium, Chryseomonas, Citrobacter, Clostridium, Collinsella, Comamonas, Corynebacterium, Coxiella, Cryptobacterium, Delftia, Dermabacter, Dermatophilus, Desulfomonas, Desulfovibrio, Dialister, Dichelobacter, Dolosicoccus, Dolosigranulum, Edwardsiella, Eggerthella, Ehrlichia, Eikenella, Empedobacter, Enterobacter, Enterococcus, Erwinia, Erysipelothrix, Escherichia, Eubacterium, Ewingella, Exiguobacterium, Facklamia, Filifactor, Flavimonas, Flavobacterium, Francisella, Fusobacterium, Gardnerella, Globicatella, Gemella, Gordona, Haemophilus, Hafnia, Helicobacter, Helococcus, Holdemania, Ignavigranum, Johnsonella, Kingella, Klebsiella, Kocuria, Koserella, Kurthia, Kytococcus, Lactobacillus, Lactococcus, Lautropia, Leclercia, Legionella, Leminorella, Leptospira, Leptotrichia, Leuconostoc, Listeria, Listonella, Megasphaera, Methylobacterium, Microbacterium, Micrococcus, Mitsuokella, Mobiluncus, Moellerella, Moraxella, Morganella, Mycobacterium, Mycoplasma, Myroides, Neisseria, Nocardia, Nocardiopsis, Ochrobactrum, Oeskovia, Oligella, Orientia, Paenibacillus, Pantoea, Parachlamydia, Pasteurella, Pediococcus, Peptococcus, Peptostreptococcus, Photobacterium, Photorhabdus, Plesiomonas, Porphyrimonas, Prevotella, Propionibacterium, Proteus, Providencia, Pseudomonas, Pseudonocardia, Pseudoramibacter, Psychrobacter, Rahnella, Ralstonia, Rhodococcus, Rickettsia Rochalimaea Roseomonas, Rothia, Ruminococcus, Salmonella, Selenomonas, Serpulina, Serratia, Shewenella, Shigella, Simkania, Slackia, Sphingobacterium, Sphingomonas, Spirillum, Staphylococcus, Stenotrophomonas, Stomatococcus, Streptobacillus, Streptococcus, Streptomyces, Succinivibrio, Sutterella, Suttonella, Tatumella, Tissierella, Trabulsiella, Treponema, Tropheryma, Tsakamurella, Turicella, Ureaplasma, Vagococcus, Veillonella, Vibrio, Weeksella, Wolinella, Xanthomonas, Xenorhabdus, Yersinia, and Yokenella; Gram-positive bacteria such as, M. tuberculosis, M. bovis, M. typhimurium, M. bovis strain BCG, BCG substrains, M. avium, M. intracellulare, M. africanum, M. kansasii, M. marinum, M. ulcerans, M. avium subspecies paratuberculosis, Staphylococcus aureus, Staphylococcus epidermidis, Staphylococcus aqui, Streptococcus pyogenes, Streptococcus agalactiae, Listeria monocytogenes, Listeria ivanovii, Bacillus anthracis, B. subtilis, Nocardia asteroides, Actinomyces israelii, Propionibacterium acnes, and Enterococcus species and Gram-negative bacteria such as Clostridium tetani, Clostridium perfringens, Clostridium botulinum, Pseudomonas aeruginosa, Vibrio cholerae, Actinobacillus pleuropneumoniae, Pasteurella haemolytica, Pasteurella multocida, Legionella pneumophila, Salmonella typhi, Brucella abortus, Chlamydi trachomatis, Chlamydia psittaci, Coxiella bumetti, Escherichia coli, Neiserria meningitidis, Neiserria gonorrhea, Haemophilus influenzae, Haemophilus ducreyi, Yersinia pestis, Yersinia enterolitica, Escherichia coli, E. hirae, Burkholderia cepacia, Burkholderia pseudomallei, Francisella tularensis, Bacteroides fragilis, Fusobascterium nucleatum, and Cowdria ruminantium. Other types of bacteria are contemplated.

In some embodiments, the microorganism comprises a bacterium of the genus Staphylococcus, or a small colony variant or antimicrobial resistant variant thereof. In some embodiments, the microorganism comprises Staphylococcus aureus or Staphylococcus epidermidis or a small colony variant or antimicrobial resistant variant thereof. In some embodiments, the microorganism comprises methicillin-resistant Staphylococcus aureus (MRSA) and therefore the bacterial infection is due to MRSA.

In some embodiments of the third aspect of the invention, the bacterial infection forms part of a biofilm. A biofilm is a cluster of bacterial cells, irreversibly attached to a surface and embedded in a matrix of extracellular polymeric substances self-produced by the bacteria. Clinically relevant biofilms are often microbial complex structures associated with severe and recalcitrant diseases, including chronic wounds, cystic fibrosis, and chronic rhinosinusitis. Staphylococcus aureus represents one of the most notorious bacteria causing invasive, superficial, chronic and nosocomial (including methicillin resistant S. aureus) infections.

The biofilm state is advantageous for bacterial survival as the biofilm acts like a protective shield, enabling the bacteria to adapt to hostile environmental conditions, evade the immune system, and ultimately to establish resistance against antibacterial agents. Indeed, bacteria residing in biofilms can require up to 1000-fold higher concentrations of an antibacterial agent for their treatment than their planktonic (free-floating) counterparts. Therefore, bacterial biofilms represent one of the biggest challenges the medical community is facing. Indeed, recent data suggest that biofilms may account for over 80% of microbial infections in the body.

Examples of bacterial infections associated with biofilms include bacterial biofilms associated with urinary tract infections (e.g. E. coli, Pseudomonas aeruginosa, enterococci, Klebsiella, Enterobacter spp Proteus, Serratia), such as being responsible for persistent infections causing relapses and acute prostatitis, wounds including acute or chronic wounds (e.g. S. aureus, P. aeruginosa), lung infections (e.g. P. aeruginosa, such as occurs in patients with cystic fibrosis), chronic osteomyelitis (e.g. S. aureus), rhinosinusitis (e.g. S. aureus), tuberculosis (e.g. M. tuberculosis) and infections associated with foreign bodies inserted in the body (e.g. S. aureus).

As indicated above, the Applicant has found that a composition of the present invention provides significant and unexpected advantages with respect to the delivery of active substances to subjects in need thereof. A composition encompassed by the present invention enables efficient delivery of agents such as poorly water-soluble drugs, including antimicrobial agents, to microbial cells and biofilms. In effect, a composition encompassed by the present invention displays an enhanced effect at treating or preventing microbial infections, including those infections present in a biofilm or planktonic state, when compared to the use of the antimicrobial agent alone.

Therefore, in such microbial infection environments, the activity of antimicrobial agent is effectively potentiated. As used herein, to “potentiate” the activity should be taken to mean to increase the activity of the antimicrobial agent to a level which is greater than the activity of the antimicrobial agent when administered alone. The activity of the antimicrobial agent may be increased by at least 1%, 5%, 10%, 20%, 30%, 40%, 50%, 60%, 70%, 80%, 90%, 100%, or greater, or by 1-fold, 2-fold, 3-fold, 4-fold, 5-fold, 6.0-fold, 7-fold, 8-fold, 9-fold, 10-fold, 15-fold, 20-fold, 25-fold, 30-fold, 35-fold, 40-fold, 45-fold, 50-fold, 60-fold, 70-fold, 80-fold, 90-fold, 100-fold, 125-fold, 150-fold, 175-fold, 200-fold, 225-fold, 250-fold, 275-fold, 300-fold, 400-fold, 500-fold, 1000-fold, 10,000-fold, 100,000-fold, or greater, when compared to the activity of the antimicrobial agent when administered alone.

Methods for measuring the activity of a composition comprising the antimicrobial agent would be well known in the art. For example, the activity may be reflective of the measured minimum inhibitory concentration (MIC), minimum bactericidal concentration (MBC), and/or minimum biofilm inhibitory concentration (MBIC) of the composition, or of the short-kill assay times with respect to an in vitro analysis. For example, a composition of the present invention comprising an antimicrobial agent may decrease the MIC, MBC, and/or MBIC of the antimicrobial agent, or reduce the short-kill time for bacteria which are resistant or tolerant to the antimicrobial agent when administered alone. The activity may also be observed in the form of an improvement of the condition of the subject, for example, as determined by a clinician.

Methods for determination of MICs, MBCs and MBICs would be well known in the art. The MIC is defined as the lowest concentration of composition that is bacteriostatic (i.e. prevents the visible growth of bacteria). MICs are used to evaluate the antimicrobial efficacy of a composition by measuring the effect of decreasing concentrations of the composition over a defined period in terms of inhibition of microbial population growth. For example, the MIC can be determined by the broth microdilution method as described in Wiegand I et al., 2008, Nature Protocols, 3(2): 163-175; and (CLSI), C.a.L.S.I., Methods for dilution antimicrobial susceptibility tests for bacteria that grow aerobically, 2012, Wayne, PA, USA. MIC values for various antibiotics and bacteria can also be obtained from the Antimicrobial Index at http://antibiotics.toku-e.com.

The MBC is the lowest concentration of a composition comprising the antimicrobial agent required to kill a bacterium over a fixed period, such as 18 hours or 24 hours, under a specific set of conditions. It can be determined from the broth dilution of MIC tests by subculturing to agar plates that do not contain the test agent. The MBC is identified by determining the lowest concentration of the composition that reduces the viability of the initial bacterial inoculum by a pre-determined reduction such as ≥99.9%. The MBC is complementary to the MIC; whereas the MIC test demonstrates the lowest level of antimicrobial agent that greatly inhibits growth, the MBC demonstrates the lowest level of antimicrobial agent resulting in microbial death. In other words, if a MIC shows inhibition, plating the bacteria onto agar might still result in organism proliferation because the antimicrobial did not cause death. The MBC can be determined by methods such as those found in CLSI M26-A, Methods for Determining Bactericidal Activity of Antimicrobial Agents, 1999, volume 19, number 18 https://clsi.org/media/1462/m26a_sample.pdf.

The MBIC is the lowest concentration of a composition comprising the antimicrobial agent required to inhibit the formation of biofilm. It can be measured using a number of assays such as microplate-based assays as described in Stepanovic S et al., 2007, Acta. Path. Micro. Im. A., 13: 891-899. Models of biofilms may also be employed to test the MBIC (for example see Macia M D et al., 2014, Clin. Microbial. Infection, 20(10): 981-990). Biofilm growth models have been classified as closed systems (batch culture) and open systems (continuous culture) (McBain A J, 2009, “Chapter 4: in vitro biofilm models: an overview”. Adv. Appl. Microbiol., 2009; 69: 99-132). Closed models have the advantage of simplicity and applicability in high-throughput analysis, whereas open models allow better control of growth parameters and dynamics (Lourenco A et al., 2014, Pathog. Dis., 70: 250-256).

Examples of closed systems for measuring MBIC include the microtitre plate method. The microtitre plate (e.g. 96-well plate) filled with sterile broth culture (depending on the type of microorganism) is inoculated with bacteria and incubated for 24 to 48 h with an appropriate atmosphere and temperature. Biofilm formation takes place as a ring around the well. After rinsing of wells to remove planktonic cells, the biofilm can be stained with crystal violet and dissolved in acetone-ethanol for quantification of the biomass by measuring the optical density (Christensen G D et al., 1985, J. Clin. Microbiol., 22: 996-1006). The main advantages are the ease, rapidity and reproducibility of the method.

The Calgary biofilm device (also known as the MBEC© device) is another example of a closed system for measuring MBIC. This device is a disposable 96-well microtitre plate with a lid that incorporates the same number of removable polystyrene pegs (Ceri H et al., 1999, J. Clin. Microbiol., 37: 1771-1776). The bacteria are inoculated in the microtitre wells with broth culture, and the plate is incubated with or without (Mulet X et al., 2009, Antimicrob. Agents. Chemother., 53: 1552-1560) shaking to allow cells to attach to pegs. The biofilm is formed around the pegs, while planktonic bacteria remain in the broth. To facilitate the growth of bacteria, the pegs can be coated with a substance, such as L-lysine or hydroxyapatite.

Open systems for measuring MBIC try to replicate the in vivo conditions through the control of nutrient delivery, flow, and temperature. Moreover, these systems make possible the implementation of pharmacokinetic/pharmacodynamic (PK/PD) models, as well as allowing observation by microscopy. Another advantage is the study of biofilm dynamics in the absence of planktonic cells (eliminated by flow). Examples of open systems for measuring MBIC include the flow cell system which has been demonstrated to be the best approach for modelling biofilm formation, as real-time non-destructive confocal laser scanning microscopy (CLSM) analyses can be performed (Klausen M et al., 2003, Mol. Microbiol., 48: 1511-1524). The system includes a vessel with sterile broth culture that provides medium through a multi-channel peristaltic pump. The bacteria are directly inoculated into the flow cells by injection through silicone tubing. Cells are attached to a surface, where biofilm starts to develop. The most common attachment surfaces used are transparent and non-fluorescent microscope coverslips, in order to allow biofilm evolution to be observed. Another advantage is that a defined constant environment is provided by laminar flow (Palmer R J Jr., 1999, Methods Enzymol., 310: 160-166). In addition, biofilms formed in this model are thicker than those obtained with the Calgary biofilm device.

The suspended substratum reactor (CDC Biofilm Reactor) is another example of an open system for measuring MBIC. This system consists of a glass reactor connected to a flask with sterile broth culture, which is pumped through the system. Eight coupon holders, each one housing three coupons (diameter, 12.7 mm; surface area, 2.53 cm2), are suspended from a lid placed into the reactor filled with growth medium. The bacteria are inoculated into the reactor, and the biofilm is formed upon coupons while the broth is mixed with a stirring vane by magnetic rotation. Owing to the rotation, the biofilm grows under high-shear conditions. Coupons can be sampled by removing individual coupon holders and replacing them in the lid to continue the experiment in aseptic conditions. These coupons can be made from a large number of materials (polycarbonate, mild steel, stainless steel, PVC, vinyl, glass, etc), according to the microorganism and assay. The conditions of the experiments can be controlled by modifying the flow speed, temperature, and residence times. This method allows the study of seeding planktonic cells by sampling the bulk fluid phase.

In some embodiments, the MIC, MBC or MBIC of a composition comprising the antimicrobial agent may be reduced by at least 10%, 20%, 30%, 40%, 50%, 60%, 70%, 80%, 90%, 100%, or greater, or by 10-fold, 20-fold, 30-fold, 40-fold, 50-fold, 60-fold, 70-fold, 80-fold, 90-fold, 100-fold, 200-fold, 300-fold, 400-fold, 500-fold, or greater, compared to administration of the antimicrobial agent alone. Other amounts are contemplated.

In some embodiments, the bacterial infection comprises an infected wound. Examples of wounds include acute wounds (such as those caused by abrasions, cuts and more serious penetrative injuries, burns, abscesses, nerve damage and wounds resulting from elective surgery), chronic wounds (such as diabetic, venous and decubitus ulceration) or wounds in individuals with compromised wound healing capacity, such as the elderly. In some embodiments, the bacterial infection comprises a post-surgery infected wound, for example an infected wound following abdominal surgery or sinus surgery.

Methods for assessing bacterial infection are known in the art. For example, bacterial infection in a wound would delay healing of the wound. As such various wound healing assays commonly known in the art could be utilised to test for assessing bacterial infection associated with wounds and healing thereof. One such assay is the scratch wound assay where a “wound gap” in a cell monolayer (such as a fibroblast or keratinocyte monolayer) is created by scratching, and the “healing” of this gap by cell migration and growth towards the centre of the gap is monitored and often quantified. Factors such as bacterial infection can alter the motility and/or growth of the cells which leads to a decreased rate of “healing” of the gap. An exemplary scratch wound assay can be found in Chen Y, 2012, Bio-protocol 2(5): e100. Other commonly used wound assays can be found in Kopecki W et al., 2017, Wound Practice and Research, 25(1): 6-13.

The terms “treat”, “treating” or “treatment,” as used herein are to be understood to include within their scope obtaining a desired pharmacologic and/or physiologic effect in terms of improving the condition of the subject. This may be measured by one or more of the following non-limiting outcomes: (i) inhibiting to some extent the growth of a microorganism which is causing an infection in the subject, including, slowing down or complete growth arrest of the microorganism; (ii) inhibiting to some extent the growth and/or formation of one or more secondary microorganism infections in the subject; (iii) improving the life expectancy of the subject as compared to the untreated state; (iv) improving the quality of life of the subject as compared to the untreated state; (v) alleviating, abating, arresting, suppressing, relieving, ameliorating, and/or slowing the progression of at least one symptom caused by a microorganism infection in the subject; (vi) a partial or complete stabilization of the subject; (vii) a regression of one or more symptoms in the subject; and (viii) a cure of a disease, condition or state in the subject.

The terms “prevent”, “preventing” and “prevention” as used herein are to be understood to include within their scope obtaining a desired pharmacologic and/or physiologic effect in terms of arresting or suppressing the appearance of one or more symptoms in the subject. For example, inhibiting the formation of a microorganism infection in the subject.

As used herein, the term “subject” should be taken to encompass any subject which would benefit from administration of the composition of the present invention. In some embodiments, the subject is a human or animal subject. The animal subject may be a mammal, a primate, a livestock animal (e.g. a horse, a cow, a sheep, a pig, or a goat), a companion animal (e.g. a dog, a cat), a laboratory test animal (e.g. a mouse, a rat, a guinea pig, a bird), an animal of veterinary significance, or an animal of economic significance.

In a fourth aspect, the present invention provides a method for administering an active substance to a subject, wherein the method comprises administering to the subject a composition (which comprises the active substance) according to the first aspect of the present invention.

In a fifth aspect, the present invention provides a method for producing a composition of the first aspect of the invention, wherein the method comprises spray-drying a lipid-in-water nano-emulsion comprising the lipid droplets and the polysaccharide. This may be achieved using the methods described above.

In a sixth aspect, the present invention provides a dry composition comprising:
(i) a polysaccharide;
(ii) lipid droplets; and
(iii) an excipient or stabilizer,
wherein the lipid droplets are encapsulated within polymeric chains of the polysaccharide, and wherein the polysaccharide is not in a nano-particulate form.

In some embodiments, the stabilizer is lecithin, as described above. In some embodiments, the composition according to the sixth aspect of the invention further comprises an active substance, as described above, wherein the active substance is contained within the lipid droplets. For example, the active substance is an agent that can dissolve in the lipid droplets, as described above.

The composition of the sixth aspect of the invention may be produced by a 2-step method as described above. For example, in a first step the method comprises producing a lipid-in-water nano-emulsion comprising the lipid droplets, the polysaccharide, and the excipient or stabilizer, wherein the polysaccharide is dissolved in the aqueous phase. A method for producing the lipid-in-water nano-emulsion has been described above. For example, by homogenizing a mixture comprising the lipid droplets and the excipient or stabilizer, and adding the polysaccharide in aqueous form to the mixture. In a second step, the nano-emulsion is spray-dried using methods as described above.

In a seventh aspect, the present invention provides a method for improving the health of a subject, or for treating or preventing a disease or disorder in a subject, the method comprising administering a composition according to the sixth aspect of the invention to the subject. Details regarding health improvement, disease or disorder treatment or prevention and composition administration are as described above.

In some embodiments where the composition of the sixth aspect of the invention comprises an active substance which is an antimicrobial agent, the composition can be used to treat or prevent a microbial infection in a subject. Accordingly, in an eighth aspect, the invention provides a method for treating or preventing a microbial infection in a subject, the method comprising administering the composition comprising the antimicrobial agent to the subject. Details regarding antimicrobial agents, and microbial infection treatment or prevention are as described above.

In a ninth aspect, the present invention provides a method for administering an active substance to a subject, wherein the method comprises administering to the subject a composition (which comprises the active substance) according to the sixth aspect of the present invention.

In a tenth aspect, the present invention provides a method for producing a composition of the sixth aspect of the invention, wherein the method comprises spray-drying a lipid-in-water nano-emulsion comprising the lipid droplets, the polysaccharide, and the excipient or stabilizer. This may be achieved using the methods described above.

In an eleventh aspect, the present invention provides a dry composition comprising:
(i) inulin;
(ii) an active substance; and
(iii) lipid droplets,
   wherein the lipid droplets are encapsulated within polymeric chains of inulin, and wherein the polysaccharide is not in a nano-particulate form.

In some embodiments, the composition according to the eleventh aspect of the invention further comprises an active substance, as described above, wherein the active substance is contained within the lipid droplets. For example, the active substance is an agent that can dissolve in the lipid droplets, as described above.

The composition of the eleventh aspect of the invention may be produced by a 2-step method as described above. For example, in a first step the method comprises producing a lipid-in-water nano-emulsion comprising the lipid droplets, the active substance, and the inulin, wherein the inulin is dissolved in the aqueous phase. A method for producing the lipid-in-water nano-emulsion has been described above. For example, by homogenizing a mixture comprising the lipid droplets and the active substance, and adding the inulin in aqueous form to the mixture. In a second step, the nano-emulsion is spray-dried using methods as described above.

In a twelfth aspect, the present invention provides a method for treating or preventing a disease or disorder in a subject, the method comprising administering a composition according to the eleventh aspect of the invention to the subject. Details regarding health improvement, disease or disorder treatment or prevention and composition administration are as described above.

In some embodiments where the composition of the eleventh aspect of the invention comprises an active substance which is an antimicrobial agent, the composition can be used to treat or prevent a microbial infection in a subject. Accordingly, in a thirteenth aspect, the invention provides a method for treating or preventing a microbial infection in a subject, the method comprising administering the composition comprising the antimicrobial agent to the subject. Details regarding antimicrobial agents, and microbial infection treatment or prevention are as described above.

In a fourteenth aspect, the present invention provides a method for administering an active substance to a subject, wherein the method comprises administering to the subject a composition according to the eleventh aspect of the present invention.

In a fifteenth aspect, the present invention provides a method for producing a composition of the eleventh aspect of the invention, wherein the method comprises spray-drying a lipid-in-water nano-emulsion comprising the lipid droplets, the active substance, and the inulin. This may be achieved using the methods described above.

In further aspects, the present invention provides compositions comprising various combinations of components, as described above, and uses of said compositions. For example, a dry composition is provided, comprising:
(i) inulin;
(ii) lipid droplets; and
(iii) lecithin,
   wherein the lipid droplets are encapsulated within polymeric chains of the inulin, and wherein the inulin is not in a nano-particulate form.

A method for improving the gastrointestinal health of a subject is also provided, the method comprising administering to the subject a dry composition comprising:
(i) inulin;
(ii) lipid droplets; and
(iii) lecithin,
   wherein the lipid droplets are encapsulated within polymeric chains of the inulin, and wherein the inulin is not in a nano-particulate form.

A method for treating or preventing a metabolic disorder or disease in a subject is also provided, the method comprising administering to the subject a dry composition comprising:
(i) inulin;
(ii) lipid droplets; and
(iii) lecithin,
   wherein the lipid droplets are encapsulated within polymeric chains of the inulin, and wherein the inulin is not in a nano-particulate form.

A dry composition is provided comprising:
(i) inulin;
(ii) lipid droplets; and
(iii) a poorly water-soluble active substance,
   wherein the lipid droplets are encapsulated within polymeric chains of the inulin, and wherein the inulin is not in a nano-particulate form.

A method for treating or preventing a disease or disorder in a subject is also provided, the method comprising administering to the subject a dry composition comprising:
(i) inulin;
(ii) lipid droplets; and
(iii) a poorly water-soluble active substance,
   wherein the lipid droplets are encapsulated within polymeric chains of the inulin, and wherein the inulin is not in a nano-particulate form.

A dry composition is provided comprising:
(i) inulin;
(ii) lipid droplets; and
(iii) an antibiotic,
   wherein the lipid droplets are encapsulated within polymeric chains of the inulin, and wherein the inulin is not in a nano-particulate form.

A method for treating or preventing a bacterial infection in a subject is also provided, the method comprising administering to the subject a dry composition comprising:
(i) inulin;
(ii) lipid droplets; and
(iii) an antibiotic,
   wherein the lipid droplets are encapsulated within polymeric chains of the inulin, and wherein the inulin is not in a nano-particulate form.

The amount of each component (e.g. lipid, polysaccharide, active substance, excipient or stabilizer, etc) in each composition of the invention described herein will vary depending on factors which have been described above—for example the nature of the polysaccharide, physical and chemical properties of the active substance, the type of lipid used, and other factors specific to the subject ultimately being administered the composition. Representative amounts of each component have been described above.

Accordingly, in some embodiments a composition of the present invention may comprise the following:
about 10 wt % to about 90 wt % of polysaccharide (preferably about 50 wt % to about 70 wt %); and about 10 wt % to about 95 wt % of lipid (preferably about 30 wt % to about 50 wt %);
about 10 wt % to about 90 wt % of polysaccharide (preferably about 50 wt % to about 70 wt %); about 10 wt % to about 95 wt % of lipid (preferably about 30 wt % to about 50 wt %); and about 0.05 wt % to about 10 wt % of an excipient or stabilizer;
about 10 wt % to about 90 wt % of inulin (preferably about 50 wt % to about 70 wt %); about 10 wt % to about 95 wt % of lipid (preferably about 30 wt % to about 50 wt %); and about 0.05 wt % to about 10 wt % of lecithin;
about 10 wt % to about 90 wt % of polysaccharide (preferably about 50 wt % to about 70 wt %); about 10 wt % to about 95 wt % of lipid (preferably about 30 wt % to about 50 wt %); and about 0.5 wt % to about 25 wt % of an active substance;
about 10 wt % to about 90 wt % of inulin (preferably about 50 wt % to about 70 wt %); about 10 wt % to about 95 wt % of lipid (preferably about 30 wt % to about 50 wt %); and about 0.5 wt % to about 25 wt % of an active substance;
about 10 wt % to about 90 wt % of inulin (preferably about 50 wt % to about 70 wt %); about 10 wt % to about 95 wt % of lipid (preferably about 30 wt % to about 50 wt %); and about 0.5 wt % to about 25 wt % of a poorly water-soluble active substance; and
about 10 wt % to about 90 wt % of inulin (preferably about 50 wt % to about 70 wt %); about 10 wt % to about 95 wt % of lipid (preferably about 30 wt % to about 50 wt %); and about 0.5 wt % to about 25 wt % of an antibiotic.

Other combinations of components for the composition are contemplated and encompassed by the present invention.

The invention is further illustrated in the following examples. The examples are for the purpose of describing particular embodiments only and are not intended to be limiting with respect to the above description.

### Example 1

### Composition for the Delivery of an Antibiotic to Infected Macrophages

In this study, the poorly soluble and poorly cell membrane permeable antibiotic, rifampicin, was re-purposed via microencapsulation within polysaccharide-lipid hybrid particles for the treatment of macrophages infected with small colony variants of Staphylococcus aureus (SCV S. aureus).

The over-consumption and over-prescribed application of antibiotics has triggered the emergence of drug-resistant bacteria that have evolved an array of evasive mechanisms. For example, in 2011, the World Health Organization (WHO) reported that among the 12 million worldwide cases of tuberculosis, 630,000 of these patients have developed a multi-drug resistance strain. Among the various evasive mechanisms, many bacterial pathogens, including Mycobacterium tuberculosis, Staphylococcus aureus and Salmonella enterica, are now capable of being shielded within host cells, which leads to latent or recurrent infection.

Intracellular bacteria serve as a problematic source of infection due to their ability to evade biological immune responses and the inability for conventional antibiotics to efficiently penetrate cellular membranes. Consequently, new drug delivery and treatment approaches are urgently required to effectively eradicate intracellular pathogens residing within immune cells (e.g. macrophages).

Therefore, the aim of this study was to develop a drug delivery system composed of lipid nano-droplets coated with polysaccharide (in this case inulin) in a dry powder form where rifampicin was encapsulated as an antimicrobial agent. The ability of the inulin-lipid hybrid (ILH) microparticles of the drug delivery vehicle to effectively transport the drug to the site of infection was systemically investigated by monitoring drug release and uptake within the intracellular environment of macrophages and the subsequent reduction of SCV S. aureus colony forming growth.

### Materials and Methods

### Materials

Capmul MCM was obtained from Abitec Pty Ltd (NSW, Australia). Inulin from dahlia tubers, rifampicin, Nile red, phosphate-buffered saline (PBS), Dulbecco's Modified Eagle's Medium (DMEM), 4′,6-diamidino-2-phenylindole (DAPI), penicillin-streptomycin antibiotic mixture and Fetal Bovine Serum (FBS) were purchased from Sigma-Aldrich (Castle Hill, Australia). The following materials were of analytical grade and obtained from Ajax Finechem Pty Ltd., Australia: acetonitrile, acetone, dichloromethane and methanol. Murine macrophages RAW 264.7 were obtained from American Type Culture Collection (ATCC, Manassas, VA, USA). Small colony variants (SCV) of S. aureus were provided by Dr Stephen Kidd (University of Adelaide, Australia).

### Synthesis of Rifampicin-Loaded Inulin-Lipid Hybrid (ILH) Microparticles

Rifampicin-loaded ILH microparticles (Rif-ILH) were synthesised using a two-step homogenization and spray drying process. Firstly, a 2% w/v lipid-in-water emulsion was prepared by dispersing Capmul MCM (1 g) and rifampicin (100 mg) in Milli-Q water (50 mL), with stirring at 1000 rpm for 30 min at room temperature. The emulsion was ultrasonicated for a further 30 min to create a nano-emulsion. An inulin solution (2% w/v), prepared by dissolving inulin (1 g) in Milli-Q water (50 mL) with stirring and heating at 60° C. for 15 min, was added dropwise to the homogenized emulsion and stirred for another 1 h. The nano-emulsion, with inulin dissolved in the aqueous phase, was spray dried (Mini Spray Dryer B-290, Buchi Labortechnik AG) to form ILH microparticles under the following conditions: emulsion flow rate: 0.5 mL/min, air flow rate: 0.6 m³/min, inlet temperature: 160° C., outlet temperature: 90° C., and an aspirator setting of 100%.

Rifampicin-loaded lipid micro-droplets (Rif-lipid) were also prepared to be serve as a control system to Rif-ILH. To achieve this, rifampicin (100 mg) was dissolved in Capmul MCM (1 g), followed by addition of 50 ml Milli-Q water. The emulsion was then stirred at 1000 rpm for 30 min to create lipid micro-droplets. Note: No homogenization process was applied in formation of lipid micro-droplets.

### Characterization of ILH Microparticles

### Size and Zeta Potential

Particle size was evaluated by using laser diffraction (Malvern Mastersizer, Malvern Instruments, Worcestershire, UK) and zeta potential was measured using phase analysis light scattering instrument (Zetasizer Nano ZS, Malvern Instruments, Worcestershire, UK) with the laser set at a 173° fixing scattering angle. Spray dried ILH microparticles were dispersed in PBS (pH 7.4) at a concentration of 0.1 mg/mL w/v and subsequently the average particle size was measured (refractive index=0.145). All measurements were performed in triplicate.

### Scanning Electron Microscopy (SEM)

SEM (Carl Zeiss Microscopy, Oberkochen, Germany) was used to observe the morphology of the prepared ILH particles. A sample of ILH was placed on carbon tape and sputter-coated using a thin layer of platinum (˜10-20 nm) prior to imaging. The SEM imaging was performed using accelerating voltages of 1 KV.

### Quantification of Drug Loading

Rifampicin loading within ILH microparticles was determined using extraction and analysis by high performance liquid chromatography (HPLC) (UFLC XR, Shimadzu, Japan). Approximately 10 mg of powder was weighed and dissolved in 10 ml methanol: water (50:50) mixture followed by sonication for 45 minutes. Samples (1 mL) were then centrifuged for 15 min at 8944 g and the supernatants were collected and properly diluted prior to be analysed using HPLC, under the following conditions; mobile phase: mixture of methanol, phosphate buffer (pH 7.5), and acetonitrile (50:33:17%, v/v); stationary phase: C18 column (25×0.46 cm internal diameter, 5 μm pore size, Altech); flow rate: 1 mL/min; detection wavelength: 230 nm.

Assessing pH-Responsiveness of ILH Microparticles

The pH-responsive nature of ILH microparticles was evaluated in vitro within neutral media (PBS; pH 7.4) simulating the pH environment of plasma and artificial lysosomal fluid (ALF; pH 4.5) simulating the acidic environment inside macrophages. ALF was prepared according to the composition previously described in literature (Marques M et al., Simulated Biological Fluids with Possible Application in Dissolution Testing, 2011, 18: 15-28). ILH was dispersed in PBS or ALF (10 mL, 37° C.) at a concentration equivalent to 80 μg/mL rifampicin with constant stirring at 200 rpm for a 4 h period. Aliquots (500 μL) were periodically taken and were centrifuged immediately (10 min, 8944 g) to analyze the cleavage and breakdown of ILH microparticles and the extent of rifampicin release.

### In Vitro Cleavage and Breakdown of ILH Microparticles

Following centrifugation, the supernatant was neutralized by addition of saturated sodium bicarbonate solution (30 μL), to inhibit further acid-mediated hydrolysis, followed by dilution with mobile phase for determining inulin hydrolysis using HPLC analysis. That is, the concentration of fructose, glucose and sucrose cleaved from inulin particles was analysed using a HPLC system (Shimadzu Corporation, Japan) consisting of a series of LC-20ADXR pumps, SIL-20ACXR auto sampler, CTO-20AC column oven set at 30° C., and ELSD-LTII evaporative light scattering detector, and a Luna amino analytical column (NH₂, 5 μm, 4.6 mm ID×250 mm). The mobile phase was a mixture of acetonitrile and milli-Q water (95:5 v/v), eluted at a flow rate of 1.0 ml/min. The limit of detection (LOD) of the analytical method was 20 μg/ml for all sugars. Linear calibration curves (R2≥0.99) were plotted for chromatographic peak areas against sugar concentrations over the range of 33-1000 μg/ml, without the addition of an internal standard. All analytes were diluted suitably to meet the calibration concentration range.

In Vitro Rifampicin Release from ILH Microparticles

Rifampicin release was evaluated from both ILH and lipid samples, equivalent to 80 μg/mL rifampicin, were suspended in 10 mL media and incubated at 37° C. with constant rotation. At pre-determined time points, an aliquot of 1 mL sample was withdrawn and centrifuged for 10 min at 8944 g to sediment the particles. The supernatant was collected and diluted with mobile phase prior to HPLC analysis, while an equal amount of fresh media was used to re-disperse the pellet before being transferred back into vials. All samples were evaluated in triplicate.

### Cellular Cytotoxicity of ILH Microparticles

A cytotoxicity study was conducted in RAW 264.7 macrophages using the previously reported method (Maghrebi S et al., Poly(lactic-co-glycolic) Acid-Lipid Hybrid Microparticles Enhance the Intracellular Uptake and Antibacterial Activity of Rifampicin. ACS Applied Materials & Interfaces 2020, DOI: 10.1021/acsami.9b22991). 100 μL of cell suspension at a density of 7000 cells per well was seeded in 96-well plate and incubated (37° C., 5% CO₂) for 12 h to allow the attachment of cells. After incubation the supernatant was discarded and replaced with 200 μL of particle solution (in DMEM) at various concentrations (ranging from 10 to 100 μg/mL) and incubated for further 24 h. After incubation, the cells were washed with sterile PBS and 10 μL of MTT (3-(4,5-dimethylthiazol-2-yl)-2,5-diphenyltetrazolium bromide) solvent was added to each well prior to incubation for a further 4 h. Afterward, the supernatant was discarded, and the absorbance was read at OD=540 nm using a microplate reader.

### Particle Uptake Studies in Macrophages

### Uptake Studies Using Flow Cytometry

The cellular uptake of ILH particles and lipid micro-droplets was investigated using fluorescence activated cell sorting (FACS) (Maghrebi S et al., 2020, supra). Freshly grown RAW 264.7 (˜7×10⁴ cells per well), were plated in a 24-well plate and incubated for one day to promote cell adherence. After supernatant removal, the cells were exposed to either Nile red (50 μg/mL in DMEM) or Nile red loaded formulations at a final concentration equivalent to 50 μg/mL. Following 1 h and 4 h incubation, the cells were thoroughly rinsed (three times using sterile PBS) and collected from each well of plate. The cells were centrifuged at 600 g for 5 min and the subsequent cell pellets were resuspended in FACS buffer. The Nile red intensity inside cells was measured using Accuri C6 Plus flow-cytometer (BD Biosciences, Franklin Lakes, New Jersey, USA).

### Uptake Studies Using Confocal Microscopy

The uptake of particles by RAW 264.7 cells was visualized using confocal laser scanning microscope (CLSM; Zeiss Elyra PS.1 laser scanning confocal microscope, Germany). Briefly, cells were incubated overnight prior to being exposed to free Nile red, Nile red loaded lipid micro-droplets and ILH at a Nile red concentration of 50 μg/mL for 1 h. After incubation and prior to staining the nuclei and cytoskeleton with 4′,6-Diamidino-2-Phenylindole (DAPI) and Alexa-488, respectively, the cells were rinsed with PBS three times and fixed with paraformaldehyde (PFA) (4%) for 20 min. DAPI with the emission wavelength of 461 nm (excitation wavelength 358 nm) and Alexa-488 with 525 nm emission wavelength (excitation wavelength 490 nm) appeared as blue and green, respectively.

Rifampicin Internalisation within Macrophages

To study the intracellular rifampicin uptake, RAW 264.7 cells at density of 5×10⁴ cells were plated in each well of a 96-well plate using fresh DMEM and incubated overnight. After incubation, the supernatant was discarded and the attached cells were washed three times with PBS, prior to treating the cells with an 80 μg/mL rifampicin dose (samples were suspended in DMEM) for 1 h and 4 h incubation periods. Following this, the cells were washed 4 times to ensure the removal of extracellular particles and lysed for 15 minutes with DMSO at 37° C., 5% CO₂. The lysed cells were then collected in an Eppendorf tube and centrifuged for 5 min at 600 g. The intracellular rifampicin uptake was evaluated by analysing the supernatant using described HPLC method.

### In Vitro Antibacterial Studies

### Intracellular Bacterial Staining

To confirm and visualize the existence of SCV S. aureus inside macrophages, RAW 264.7 cells were seeded in a 6-well culture slide at a density of 2.5×10⁵ cells per well and cultured overnight. RAW 264.7 cells were then infected with bacteria at a density of 2.5×10⁷ cells/well followed by 1 h incubation. Following incubation, macrophages were washed 4 times with PBS to remove the extracellular bacteria and then fixed with 4% PFA. Prior to imaging by confocal microscopy, the nuclei were stained with DAPI and the cell/bacteria membrane was stained with Alexa-488.

In Vitro Efficacy Studies Against Intracellular SCV S. aureus

The intracellular infection assay was performed using the method previously applied (Maghrebi S et al., 2020, supra). Briefly, a SCV S. aureus suspension in DMEM media was added to RAW 264.7 cells and incubated for 1 h, as highlighted above. The cells were subsequently washed 4 times with PBS to remove any extracellular bacteria. Infected cells were then treated with rifampicin solution (in DMEM media), rifampicin loaded ILH (Rif-ILH), and rifampicin loaded lipid micro-droplets (Rif-Lipid) at a drug concentration of 0.5 and 2.5 μg/mL for 4 h. The Colony Forming Unit (CFU) value, was then calculated based on the number of grown bacteria on a Tryptone Soy agar (TSA) plate. Results and Discussion Fabrication and Characterization of Rifampicin-loaded Inulin-Lipid Hybrid (Rif-ILH) Microparticles

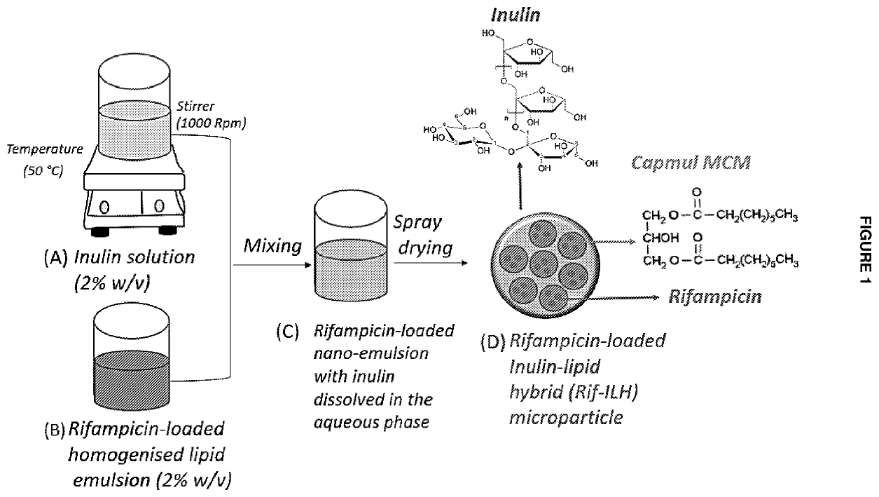

Rif-ILH microparticles of 3.1±1.1 μm mean particle size, were synthesised by spray drying rifampicin encapsulated lipid nano-droplets (droplet size=160±27 nm), comprised of medium chain length lipids dispersed within an inulin aqueous solution at a 1:1 lipid:inulin ratio (FIG. 1). Rif-ILH microparticles revealed a negative zeta potential of −11.5±0.53 mV that was attributed to deprotonation of hydroxyl groups on inulin chain in exposure to aqueous media that trigger the reduction of zeta potential (Afinjuomo F et al., Synthesis and Characterization of pH-Sensitive Inulin Conjugate of Isoniazid for Monocyte-Targeted Delivery. Pharmaceutics 2019, 11: 555, DOI: 10.3390/pharmaceutics11110555). Rifampicin loaded lipid micro-droplets (Rif-lipid), 2.4±0.8 μm in diameter with a zeta potential of −4.1±1.2 mV, were also synthesized to serve as a control system to Rif-ILH. Rifampicin loading within Rif-ILH and Rif-lipid was 5.32±0.84% (w/w) and 9.12±1.10% (w/w), respectively.

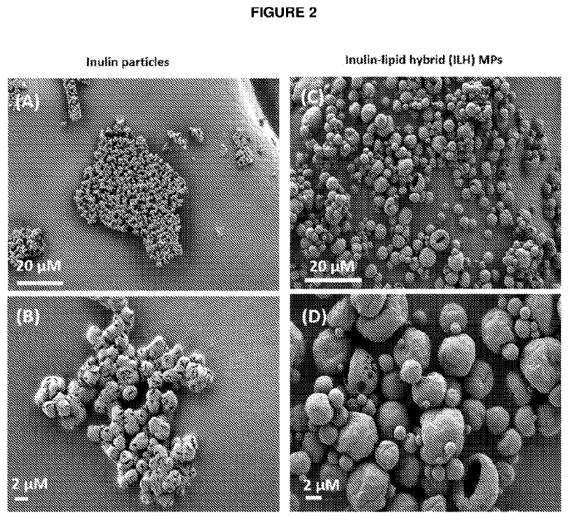

The morphology of powdered inulin particles and spray dried Rif-ILH microparticles was investigated using scanning electron microscopy (SEM). As shown in FIG. 2, raw inulin powder comprised 1-2 μm particles with a spherulite-like discoid shape, which consists of stacks of lamellar sheets, according to previous investigations (Kaufmann S H E., Immunity to intracellular bacteria. In Annual Review of Immunology, 1993; 11: 129-163). In contrast, Rif-ILH microparticles were approximately 2-5 μm in diameter and exhibited a spherical shape and a smooth surface morphology. Without wishing to be bound by theory, it is hypothesised that inulin accumulated at the surface of Rif-ILH particles, with lipid nano-droplets being encapsulated within a three-dimensional inulin matrix.

Previous studies have revealed that the particle size is one of the major contributing factors for cellular uptake (Win K Y and Feng S S, Effects of particle size and surface coating on cellular uptake of polymeric nanoparticles for oral delivery of anticancer drugs, Biomaterials 26,(15): 2713-2722). The particle size of the fabricated Rif-ILH microparticles was considered ideal for this study, since previous studies have highlighted the greater ability of intermediate particles size (˜2-3 μm in diameter) to be engulfed by macrophages, compared to submicron particles and particles exceeding 10 μm (He C et al., 2010, Effects of particle size and surface charge on cellular uptake and biodistribution of polymeric nanoparticles, Biomaterials, 31(13): 3657-3666). Subsequently, it was hypothesized that the Rif-ILH microparticles prepared in this study will also promote greater uptake into macrophages. Although it is recognised that further optimisation may be required for the production of a more uniform particle size distribution and therefore greater enhancements in particle internalisation within macrophages in key target tissues associated with intracellular infection, such as the lungs.

### In Vitro Rifampicin Release Studies

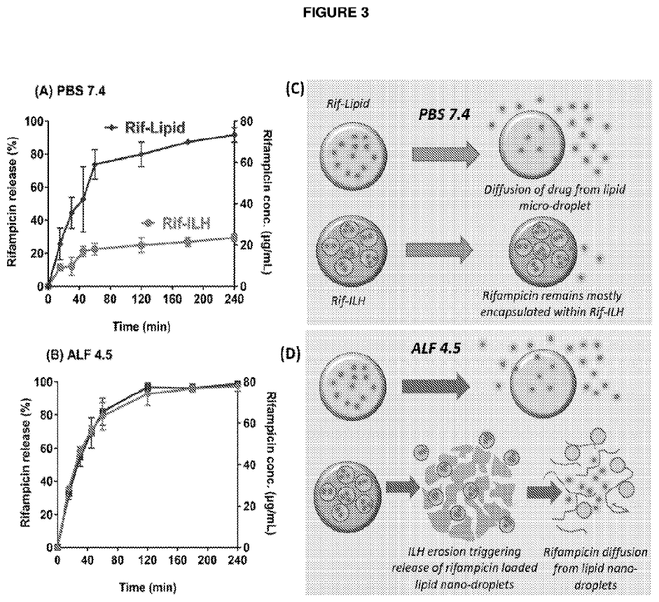

To validate the pH-responsive release behaviour of rifampicin when encapsulated within ILH microparticles, release studies were performed in the following media: (i) PBS (pH 7.4), which mimicked the neutral environments that particles are exposed to prior to cellular uptake (i.e. plasma), and (ii) artificial lysosomal fluid (ALF; pH 4.5), which simulated the acidic environment that particles are exposed to when internalised within macrophages through phagocytic pathways (Butler M S and Cooper M A, 2011, Antibiotics in the clinical pipeline in 2011. Journal of Antibiotics, 64(6): 413-425, DOI: 10.1038/ja.2011.44). In neutral media, only 22.5±3.6% of rifampicin was released from Rif-ILH particles after 1 h, in contrast to 59.0±7.1% for Rif-Lipid (FIG. 3A). Moreover, rifampicin release continued to increase for Rif-Lipid over the 4 h release period, leading to 73.4±3.5% release. Restricted rifampicin release was observed for Rif-ILH microparticles throughout the entire 4 h period, whereby only 29.4±2.2% of rifampicin partitioned towards the aqueous media (FIG. 3A).

The pH dependent release mechanism of Rif-ILH microparticles was exhibited in ALF at pH 4.5, where over three-quarters of the rifampicin was released after 1 h, with a total release of 79.1±0.2 μg/mL being observed after 4 h. For Rif-Lipid, the change in pH did not induce any significant differences in rifampicin release kinetics (FIG. 3B). These findings confirm that the inulin coating protects the majority of encapsulated drug from premature release within plasma-simulating media, since it is stable to hydrolysis in aqueous systems at room temperature and neutral pH. However, once exposed to acidic ALF media, inulin chains rapidly decompose into oligosaccharide and monosaccharide units, which triggers the diffusion of rifampicin from the exposed lipid nano-droplets. This is in accordance with previous findings that have shown the pH-provoked release mechanism of drugs conjugated to inulin chains, whereby in neutral media the drug-inulin conjugate is stable, but once exposed to an environment simulating lysosomal media the drug is cleaved from the inulin chain due to inulin hydrolysis. For example, inulin was used as a carrier for the delivery of the anti-tuberculosis (TB) drug, isoniazid, to monocytes, where inulin conjugated isoniazid exhibited a pH dependent release with less than 10% release in pH 7.4 and >40% drug release in pH 4.5 (Petrovsky N, 2010, Inulin—a versatile polysaccharide: use as food chemical and pharmaceutical agent, Journal of Excipients and Food Chemicals, 1(3): 27-50).

### Cytotoxicity of Particles Towards RAW 264.7 Cells

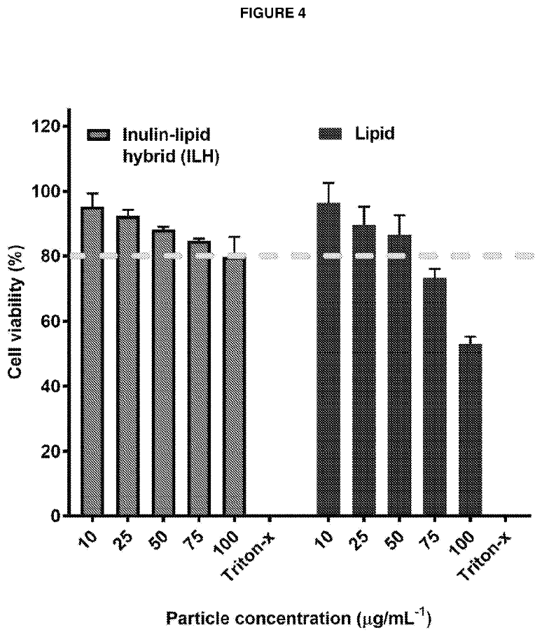

The cytotoxicity of ILH microparticles and lipid micro-droplets was evaluated using the MTT assay in RAW 264.7 macrophages (FIG. 4). Triton-X was used as a positive control with no cellular viability being observed after 1 h incubation. Both ILH microparticles and lipid micro-droplets exhibited a dose dependent effect on macrophage viability, whereby dose increases from 10 to 100 μg/mL led to reduced cellular viabilities, from 95.2% to 79.7% for ILH and from 96.3% to 52.9% for lipid micro-droplets. Importantly, the presence of the inulin coating for ILH microparticles reduced the cytotoxicity of the particles with the cellular viability remaining ≥80% even at 100 μg/mL particle concentration (Coates ARM and Halls G, Antibiotics in phase II and III clinical trials. In Handbook of Experimental Pharmacology, 2012, 212: 167-183). Subsequently, these findings validate the safety of ILH microparticles towards macrophages. Cellular uptake studies were performed at a particle concentration of 50 μg/mL for both ILH microparticles and lipid micro-droplets, since at this concentration, both systems exerted a cellular viability of >80%.

### Cellular Uptake Studies

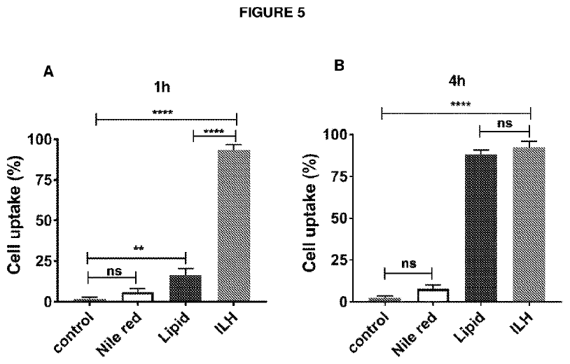

To understand whether the inulin coating of ILH microparticles promotes enhanced cellular uptake compared to uncoated lipid micro-droplets, both formulations were fluorescently labelled with Nile red (lipophilic dye) and the intracellular localization of particles was determined based on differences in fluorescence intensities within the cells, using flow cytometry. As shown in FIG. 5, both the cells incubated with Nile red solution as well as control group (no treatment) did not exhibit any significant difference in fluorescence intensity, thus confirming the inability for the pure dye to promote uptake. Subsequently, any enhancement in Nile red intensity within the cells treated with Nile red particles can be attributed to particle uptake.

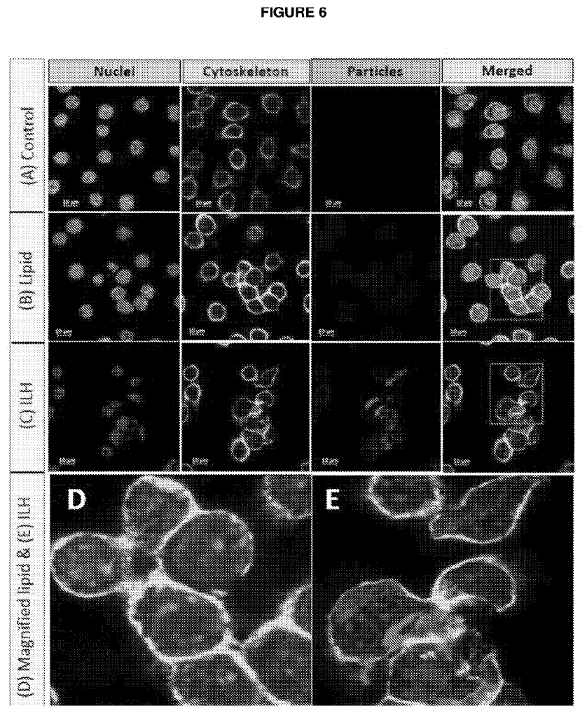

Incubation with both lipid micro-droplets and ILH microparticles triggered cellular uptake of Nile red, with 16.4±4.0% and 93.4±3.4% particles being internalised after 1 h, respectively (FIG. 5A). Thus, the degree of particle uptake for ILH microparticles was 6 to 16-fold greater compared to the lipid micro-droplet and the pure Nile red solution after 1 h incubation. This was further evidenced using confocal microscopy, whereby an enhanced dye internalisation was clearly evident for ILH particles after 1 h incubation, compared to lipid micro-droplets and pure dye (FIG. 6). No additional uptake was observed for ILH microparticles between 1 and 4 h. In contrast, lipid micro-droplets continued being internalised within macrophages during this period, achieving a total of 88.3±2.5% uptake after 4 h (FIG. 5B).

Similar outcomes were reported in earlier studies where inulin-based drug delivery systems were used for targeted intracellular antibiotic delivery to cells infected with intracellular pathogens e.g. Mycobacterium tuberculosis (Afinjuomo F et al., Design and Characterization of Inulin Conjugate for Improved Intracellular and Targeted Delivery of Pyrazinoic Acid to Monocytes. Pharmaceutics 2019, 11(5): 243; and Afinjuomo F et al., 2019, Synthesis and Characterization of pH-Sensitive Inulin Conjugate of Isoniazid for Monocyte-Targeted Delivery. Pharmaceutics, 11(11), DOI: 10.3390/pharmaceutics11110555). The results suggested the ability of inulin to promote a rapid uptake of particles by infected cells through endocytosis pathways, however the specific receptor(s) mediating this process are unknown (Counoupas C et al., 2017, Delta inulin-based adjuvants promote the generation of polyfunctional CD4+ T cell responses and protection against Mycobacterium tuberculosis infection, Scientific Reports, 7(1): 8582, DOI: 10.1038/s41598-017-09119-y; Wang L et al., 2017, Investigation of the biodistribution, breakdown and excretion of delta inulin adjuvant. Vaccine, 35(34): 4382-4388, DOI: https://doi.org/10.1016/j.vaccine.2017.06.045; and Afinjuomo F et al., 2019, supra). Ultimately, it is suggested that incorporation of inulin facilitated the rapid endocytosis of ILH microparticles by phagocytic cells, i.e. RAW 264.7 cells, leading to higher uptake of ILH microparticles compared to lipid micro-droplets in a shorter period of time.

### Rifampicin Concentration Inside RAW 264.7 Cells

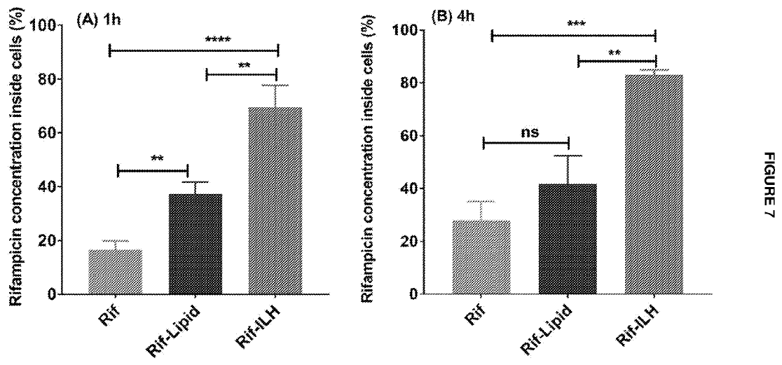

The degree of rifampicin internalisation within macrophages was examined by incubating RAW 264.7 cells with a rifampicin solution, rifampicin loaded ILH microparticles (Rif-ILH), and lipid micro-droplets (Rif-Lipid) (FIG. 7). After 1 h incubation, 37.3±4.32% and 69.5±8.17% of the rifampicin dose was detected inside the macrophages when treated with Rif-Lipid and Rif-ILH microparticles, respectively, compared to 16.5±3.26% for the cells treated with pure rifampicin solution. As the incubation time increased from 1 h to 4 h, the degree of rifampicin uptake when encapsulated within Rif-Lipid and Rif-ILH microparticles did not increase, indicating that rifampicin was internalised within the first hour of incubation. Furthermore, there was no significant difference in intracellular rifampicin concentration for the cells treated with Rif-Lipid compared to the pure drug solution after 4 h incubation, which did not correlate with the cellular uptake data obtain for the Nile red lipid micro-droplets. This can be attributed to the inability for lipid micro-droplets to protect the encapsulated cargo, specifically rifampicin, in neutral conditions equivalent to the extracellular environment. That is, drug release data indicated that over 75% of rifampicin is released from the lipid micro-droplet after 1 h at pH 7.4 (FIG. 3). Thus, after this period, lipid micro-droplets cannot facilitate uptake of rifampicin. In contrast, Rif-ILH microparticles protect the encapsulated rifampicin from premature release in the extracellular environment, until phagocytosed by the macrophages, where the acidic lysosomal media hydrolyses inulin, triggering the release of rifampicin (FIG. 3). In doing so, this highlights the important role of the pH-triggered release mechanism of Rif-ILH microparticles in transporting rifampicin to macrophages.

### Anti-Bacterial Efficacy of Rifampicin Formulations Against SCV S. Aureus

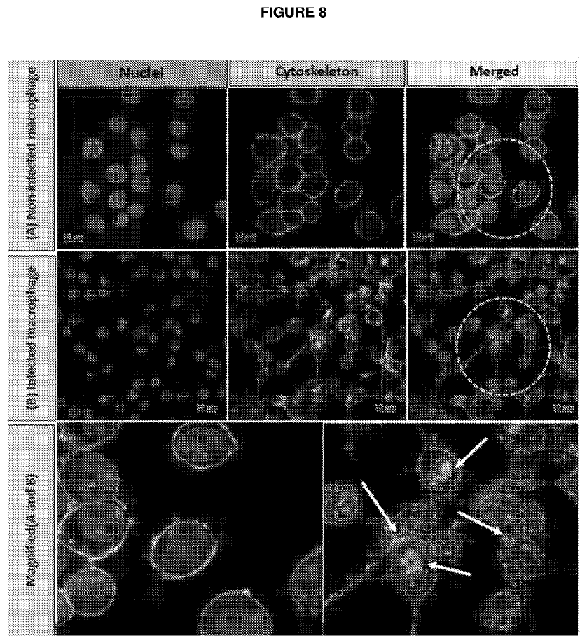

In this study, RAW 264.7 cells were infected with SCV S. aureus as a model intracellular pathogen. This pathogen is responsible for unresolved clinical problems associated with chronic infections and owing to their slow metabolism, SCV S. aureus are phenotypically smaller compared to the parent strain of S. aureus. Here, the intracellular localization of SCV S. aureus within RAW 264.7 cells was visually confirmed using confocal microscope. As illustrated in FIG. 8, the cytoskeleton of both non-infected and infected macrophages was stained green with deep purple nuclei, while the aggregation of dye (green spots) was associated with bacterial cell wall stained with Alexa 488, and highlighted the presence of SCV S. aureus within the cells.

The selection of rifampicin concentrations to evaluate the efficacy of formulations was based on the Minimum Inhibitory Concentration (MIC) against SCV S. aureus (Maghrebi S et al., 2020, Poly(lactic-co-glycolic) Acid-Lipid Hybrid Microparticles Enhance the Intracellular Uptake and Antibacterial Activity of Rifampicin, ACS Applied Materials & Interfaces, 12(7): 8030-8039), which was previously determined to be 0.125 μg/mL (Maghrebi S et al., 2020, supra). Subsequently, a rifampicin concentration equivalent to 4×MIC (0.5 μg/mL) was initially selected to ascertain the efficacy of each rifampicin formulation in triggering a reduction in intracellular bacteria. Following the 4 h treatment with rifampicin solution and Rif-Lipid, SCV S. aureus infected macrophages exhibited a negligible reduction in CFU of SCV S. aureus, which was shown to be statistically comparable to the control group (i.e., no treatment) (Error! Reference source not found.). The poor ability of unformulated rifampicin to significantly reduce the abundance of viable bacteria was anticipated since uptake studies revealed the inability for rifampicin to be internalised within macrophages. For Rif-Lipid formulation, premature drug release in neutral media reduced the exposure of intracellular pathogens to the antibiotic drug, and thus Rif-Lipid were unable to significantly improve the overall efficacy of rifampicin at 0.5 μg/mL. In contrast, treatment of infected macrophages with Rif-ILH resulted in a significant improvement in the antibacterial activity of rifampicin against SCV S. aureus. Rif-ILH demonstrated a ˜4-fold greater reduction in CFU compared to the rifampicin solution and Rif-Lipid. This correlates well with the macrophage uptake findings, where the intracellular concentration of rifampicin when hosted within Rif-ILH was over 2-times greater, compared to that within pure rifampicin and Rif-lipid. This further confirmed the increased intracellular concentration of the drug within the vicinity of the target site (i.e., site of pathogen confinement) and, therefore facilitated more efficient bacterial killing.

To evaluate a dose-dependent effect of rifampicin-loaded formulations on antibacterial activity, infected macrophages were treated with a rifampicin dose 20-fold greater than its MIC (i.e. 2.5 μg/mL) (Error! Reference source not found.). Increasing the rifampicin dose from 4×MIC to 20×MIC did not provide any further improvement in bacterial reduction for Rif-Lipid (i.e. the CFU reduced from 6.5×10⁵ CFU/mL to 2.7×10⁵ CFU/mL), while Rif-ILH microparticles showed the greatest antibacterial activity, with a ˜2-log reduction in CFU (i.e., 2×10⁵ CFU/mL to ˜2.2×10³ CFU/mL). This is in agreement with the previous study where the maximum efficacy was achieved when SCV S. aureus infected macrophages were treatment with rifampicin loaded formulations equivalent to 2.50 μg/mL rifampicin dose (Maghrebi S et al., 2020, supra).

Subsequently, this study reveals a new approach for enhanced anti-bacterial efficacy against pathogens that shield themselves in intracellular environment of the host, by encapsulating antibiotics within an inulin-based drug delivery system. Moreover, for the first time, utilizing the ILH carrier as a micro-encapsulation device enabled the advantageous effects of inulin as a delivery vehicle to be harnessed, while eliminating the need for chemical modifications to the drug via a covalent attachment mechanism. It is stipulated repurposing antibiotics using this strategy will reduce the high clinical dose required for effective bacterial killing and subsequently reduce severe side effects and toxicity associated with overconsumption of the antibiotic.

### Conclusion

A novel and efficient drug delivery system, based on hybrid microparticles of lipid and inulin, has been developed and validated for its potential application in the treatment of macrophages infected with intracellular SCV S. aureus pathogens. The synthesized hybrid microparticles demonstrated a pH-responsive release at acidic pH in artificial lysosomal fluid media, with significantly less release in neutral media, which was shown to be crucial in effectively treating pathogens located within the intracellular environment of host cells. Moreover, the inulin-lipid hybrid (ILH) microparticles were internalized efficiently by macrophages and subsequently, resulting in the enhanced intracellular release of rifampicin where bacteria are localized. The ability of the ILH system to deliver the encapsulated cargo intracellularly while avoiding the undesirable drug release prior to uptake by macrophages, was shown to be fundamental in promoting a 2-log improvement in anti-bacterial efficacy.

### Example 2

Composition for the Delivery of an Antibiotic to Staphylococcus aureus Biofilms

In this study, polysaccharide-lipid hybrid microparticles were assessed as a novel delivery system for rifampicin to treat Staphylococcus aureus biofilms.

Bacterial biofilms are linked to more than 60% of chronic infections and have been attributed to more than half of a million deaths globally each year. Bacterial biofilms are referred to as communities of aggregated planktonic bacteria embedded in a self-produced matrix of extracellular polymeric substances (EPS) which provide protection from extreme environments such as UV radiation, pH and antimicrobial agents. Infections caused by microbial biofilms are difficult to treat as they form stable complexes which are extremely tolerant to antimicrobial agents compared to planktonic cultures due to various limitations including poor solubility and permeability through the biofilm matrix and the bacterial cell membranes. This poses a significant challenge for successful antimicrobial therapy.

Rifampicin is a potent antibiotic agent often used for the treatment of infections caused by S. aureus. It inhibits the synthesis of messenger RNA by interrupting the properties of bacterial RNA polymerase that is responsible for DNA transcription. The therapeutic effectiveness of rifampicin remains challenging due to low water solubility and permeability into the bacterial cells which can result in treatment inefficiency and hepatotoxicity at high doses. Multiple in vitro studies have suggested poor responses in staphylococcal bacteria and endocarditis models after addition of rifampicin. Thus, enhancing the solubility and penetration of rifampicin through biofilms would be beneficial for antimicrobial treatment.

### Materials and Methods

The polysaccharide inulin, lecithin and rifampicin were purchased from Sigma-Aldrich (Castle Hill, Australia). Capmul MCM was obtained from Abitec Pty Ltd (NSW, Australia). Dimethyl sulfoxide (DMSO), phosphate buffered saline (PBS), tryptic soy broth (TSB), trypticase soy agar (TSA) and acetic acid were purchased from Sigma (NSW, Australia) and Staphylococcus aureus strain ATCC 25923 from American Type Culture Collection, Manassas, VA, USA.

Streak Plate Staphylococcus aureus Strain

Fresh bacteria were aseptically prepared by streaking S. aureus on a TSB plate and incubated at 37° C. for 24 h. Bacteria were transferred on a new TSB plate every week.

### Synthesis of Formulations Containing Rifampicin

### Rifampicin Solution in Dimethyl Sulfoxide (DMSO)

Rifampicin (5.8 mg) was added to DMSO (5.8 mL). Rifampicin-DMSO solution (0.16 mL) was diluted with broth (9.84 mL). This created 16 μg/mL of Rifampicin in 1.6% DMSO.

### Rifampicin-Solubilised Lipid Emulsion

A lipid emulsion was prepared by first dissolving lecithin (emulsifier) (40 mg) in Capmul MCM (1 g). Rifampicin (98 mg) was dissolved within this lipid phase, prior to emulsification in Milli-Q water (50 mL) via stirring at 1000 rpm for 30 min. The emulsion was sonicated for another 30 min at room temperature. This created 1960 μg/mL of Rifampicin-solubilised lipid nano-droplets.

### Rifampicin-Loaded Inulin-Lipid Hybrid (ILH) Microparticles

Inulin (1 g) was dissolved in Milli-Q water (50 mL) and stirred at 60° C. for 15 min (FIG. 1A). Rifampicin (100 mg) was dissolved in Capmul MCM (1 g) prior to the lipid phase being dispersed in Milli-Q water (50 mL) and stirred at 1000 rpm for 30 min at room temperature. The mixture was ultrasonicated for 30 min to form a nanoemulsion (FIG. 1B). The inulin solution was added dropwise to the rifampicin-loaded homogenised lipid emulsion and stirred for further 1 h. The nanoemulsion containing rifampicin with inulin dissolved in the aqueous phase was spray dried under the following conditions: 0.5 mL/min emulsion flow rate, 0.6 m³/min, 200° C. inlet temperature, 100° C. outlet temperature and 100% aspirator setting. This created 5.32% (w/w) of rifampicin-loaded ILH microparticles.

Minimum Inhibitory Concentration (MIC) of Rifampicin in S. aureus

Single colonies of S. aureus ATCC 25923 were suspended in 0.9% sterile saline to reach approximately 1×10⁸ colony forming units (CFU)/mL (equivalent to an optical density of 0.08-0.12 at 600 nm). The bacterial suspension was further diluted 1/100 with broth. Bacterial suspension was added to 96-wells. Serial dilution of rifampicin in 2% DMSO was added to 96-wells containing bacterial suspension (range 0.0156 μg/mL to 2 μg/mL). The 96-well plate was incubated at 37° C. for 24 h. The plates were visually assessed for turbidity indicating growth of bacteria. The lowest concentration of rifampicin did not show turbidity which was defined as the MIC of rifampicin to inhibit S. aureus growth.

### In Vitro Biofilms Studies

S. aureus from a freshly streaked TSB plate was suspended in 0.9% sterile saline and adjusted to 0.25±0.02 absorbance at 600 nm to reach approximately 3×10⁸ colony forming units (CFU)/mL. Bacterial suspension was further diluted 1/15 with broth and subsequently added to the wells of a 96-well plate. The outer wells were filled with PBS (200 μL) for hydration purposes and the second right column contained sterile broth (100 μL) to serve as negative control. The plate was wrapped in aluminium foil and incubated on a 3D rotating platform at 70 rpm at 37° C. for 24 h to grow biofilms.

After 24 h incubation, turbid biofilms sitting at the bottom of wells could be assessed visually. The broth in the wells of the plate was aspired gently by a vacuum pump without disturbing the biofilms and the wells were washed with 0.9% Saline (100 μL) twice to remove nonadherent bacteria. Rifampicin, DMSO, rifampicin-loaded DMSO, nanoemulsion, rifampicin-loaded nanoemulsion, ILH and rifampicin-loaded ILH (100 μL each) were added to the biofilms. The plate was wrapped in aluminium foil and incubated on a 3D rotating platform at 70 rpm at 37° C. for 24 h.

### Crystal Violet Assay

After 24 h of exposure to the formulations, supernatants were removed gently without disturbing the biofilms by a vacuum pump and the wells were washed with 0.9% Saline (100 μL) twice. Biofilms were fixed by oven heating at 60° C. for 1 h. The wells were subsequently exposed to 0.50% (w/v) crystal violet for 20 min and gently washed with deionized water until the water was no longer stained. The plate was dried in an oven at 60° C. for 30 min. 30% (w/v) acetic acid (120 μL) was added to each well for 15 min to solubilise the remaining dye. The biofilm biomass of S. aureus was quantified by spectrometry at 595 nm in 96 well plate reader.

### Statistical Analysis

All experiments were conducted in 6 replicates (n=6). Data were analysed as mean±standard deviation (SD). T tests and one-way analysis of variance (ANOVA) assessed the difference between biofilm treatments at a significance level of α=0.05 and 95% confidence interval.

### Results

### MIC

None of the wells showed turbidity except for positive control. Thus, the concentration of rifampicin required to inhibit growth of S. aureus strain was lower than 0.0156 μg/mL.

### Synthesis of Rifampicin-Loaded ILH Microparticles

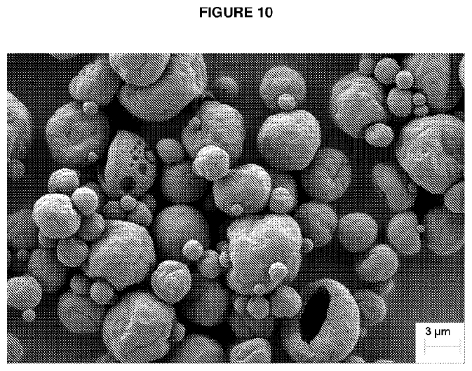

The morphology of ILH microparticles produced through spray drying was captured by scanning electron microscopy (SEM). The micrograph in FIG. 10 shows the particle size of spray dried ILH microparticles ranges from 2-5 μm. SEM imaging confirmed that spray dried ILH microparticles were spherical shape and exhibited a smooth surface morphology.

### Antimicrobial Activity Against Biofilms

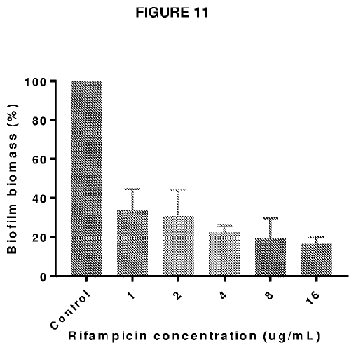

Based on the crystal violet data in FIG. 11, rifampicin in a range of 1 to 16 μg/mL showed more than 50% biofilm reduction. In addition, rifampicin at 16 μg/mL presented a trend toward greater reduction in biofilm biomass up to 82% than rifampicin at lower concentrations.

Formulations Containing Rifampicin Compared to Formulations without Rifampicin

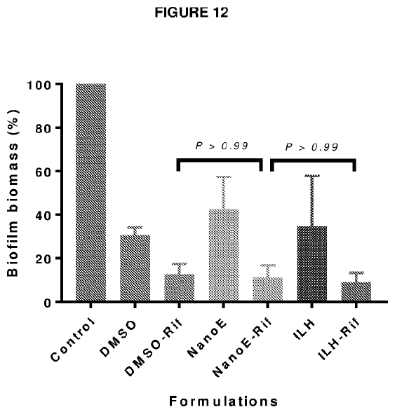

Based on the crystal violet assay, the formulations containing rifampicin (DMSO-Rif, NanoE-Rif and ILH-Rif) demonstrated similar anti-biofilm effects, with no statistical difference (P>0.99) (FIG. 12). Interestingly, formulations without rifampicin (DMSO, NanoE and ILH) showed more than 50% biofilm biomass reduction. Overall, formulations containing rifampicin showed a significantly greater reduction in biofilm biomass than treatments without rifampicin (P<0.05).

### DISCUSSION

The low water solubility and poor permeability of rifampicin following oral administration can lead to precipitation and clearance from the body, which can result in treatment inefficiency and hepatotoxicity at high doses (Subramaniam S et al., 2019, Rifampicin-Loaded Mesoporous Silica Nanoparticles for the Treatment of Intracellular Infections, Antibiotics (Basel), 8(2): 39; and Bodaghabadi N et al., 2018, Preparation and Evaluation of Rifampicin and Co-trimoxazole-loaded Nanocarrier against Brucella melitensis Infection, Iranian biomedical journal, 22(4): 275-282). Lipid-polymer hybrid microparticles can serve as an effective approach to encapsulate anti-bacterial drugs due to their ability to solubilise and increase the physicochemical stability of the therapeutic. Importantly, polymer-lipid hybrids have also shown the ability to increase the uptake and penetration of antimicrobial agents within biofilms. Polymers such as inulin can stabilise lipid nanoemulsions within a three-dimensional matrix, through the process of spray drying, generating a polymer-lipid hybrid carrier system. In this study, it was hypothesized that the antimicrobial efficacy of rifampicin against Staphylococcus aureus can be enhanced through encapsulation within inulin-lipid hybrid (ILH) microparticles. The antibiofilm performance of the ILH containing rifampicin was compared to a rifampicin solution in DMSO and rifampicin solubilised in a nanoemulsion.

The greater tolerance of biofilms against antimicrobial agents compared to planktonic bacteria was confirmed by MIC and CV assays. The MIC of S. aureus was less than 0.0156 μg/mL, which was consistent with the reported MIC in the literature (Traczewski M M et al., 1983, In vitro activity of rifampicin in combination with oxacillin against Staphylococcus aureus, Antimicrobial agents and chemotherapy, 23(4): 571-576). In contrast, S. aureus growing as biofilms were considerably more tolerant to rifampicin compared to planktonic bacteria, as evidenced by 34% of biofilm biomass remaining following exposure to 1 μg/mL of rifampicin (FIG. 11). Biofilms are more difficult to eradicate with antimicrobials compared to planktonic cells, thereby exhibiting high tolerance (Spoering A L and Lewis K, 2001, Biofilms and planktonic cells of Pseudomonas aeruginosa have similar resistance to killing by antimicrobials, Journal of bacteriology, 183(23): 6746-6751). The concentration of rifampicin selected for the following biofilm studies was based on CV assay. Since 16 μg/mL of rifampicin was the most effective in reducing biofilm biomass of S. aureus, it was selected for all formulations.

The antimicrobial efficacy of rifampicin encapsulated in ILH was compared with both DMSO and nanoemulsion containing rifampicin using the CV assay. DMSO-Rif, NanoE-Rif and ILH-Rif (formulations containing rifampicin) significantly reduced the biofilm biomass of S. aureus with 89%, 91% and 93% of biofilm reduction respectively. In contrast, significantly lower biofilm biomass reductions (P<0.05) were observed for blank carriers for DMSO (75%), NanoE (59%) and ILH (65%), respectively (FIG. 12). Despite the enhanced antimicrobial activity produced by all formulations in the presence of rifampicin, no significant difference was observed between DMSO-Rif, NanoE-Rif and ILH-Rif (P>0.99). This might be attributed to a sufficiently high solubility of rifampicin to exert antimicrobial activity even at low concentration. Interestingly, formulations in the absence of rifampicin also showed substantial biofilm biomass reduction. A plausible explanation is that DMSO is an aprotic solvent which exhibits toxicity to bacteria, thereby affecting biofilm growth (Verheijen M et al., 2019, DMSO induces drastic changes in human cellular processes and epigenetic landscape in vitro, Scientific reports, 9(1): 1-12). Similarly, both blank nanoemulsion and ILH showed biomass reductions although antibiotic was not added. Both nanoemulsion and ILH were prepared with mixed mono/di-glyceride (Capmul MCM) which has been shown to reduce the growth of bacteria in the planktonic and biofilm mode of growth.

ILH microparticles were successfully synthesized as a delivery system for antimicrobial drug rifampicin. The impact of particle size and morphology on antimicrobial activity were not investigated in this project due to limited time. Biofilm studies using S. aureus presented a substantial biofilm biomass reduction compared to different formulations containing rifampicin and formulations without rifampicin. The outcome of this research showed similar anti-biofilm effects of different formulations containing rifampicin (DMSO-Rif, NanoE-Rif and ILH-Rif). Nanoemulsion systems are commercially used in the clinic as nanocarriers for antibiotics. However, studies have suggested that the nanoemulsion systems suffer from several limitations including poor physical and chemical stability (Norouzi P et al., 2020, Nanoemulsions for intravenous drug delivery, Nanoengineered Biomaterials for Advanced Drug Delivery, pp 581-601; Date AA and Nagarsenker M S, 2008, Parenteral microemulsions: An overview, International Journal of Pharmaceutics, 355(1): 19-30). Hybrid microparticles composed of inulin and lipid can be a potential dry power formulation for rifampicin to treat biofilm infections, which may lower the risk of side effect at high doses of drugs to combat Staphylococcal infections. The expected concentration of rifampicin in nanoemulsion may have not been achieved because of storage stability concerns of rifampicin nanoemulsion as observed by orange precipitates on the wall of the storage flask.

### Conclusion

To address the limitations regarding low solubility of rifampicin for biofilm treatment, a lipid-polymer hybrid drug delivery system was synthesized where rifampicin was encapsulated within the lipid phase. Spray drying of the lipid phase with inulin generated inulin-lipid hybrid (ILH) microparticles for enhanced stability. Microparticles containing lipid and inulin in combination with rifampicin showed similar anti-biofilm effects as other formulations containing rifampicin (DMSO-Rif and NanoE-Rif). Due to the cytotoxic properties of DMSO and unstable nanoemulsion, ILH microparticles act as a dry power formulation for rifampicin to treat biofilm-related infections caused by S. aureus.

### Example 3

### Composition for Carrying a Poorly Water-Soluble Drug

In this study, the influence of polysaccharide-lipid hybrids in enhancing the in vitro solubilisation and solid-state stability of fenofibrate, a poorly water-soluble drug (PWSD), was investigated. Three formulations were prepared with different ratios of polysaccharide (in this case inulin) to lipid and measured against controls to investigate the role of polysaccharide in modulating the release of the lipid and fenofibrate.

Fenofibrate is a drug well-known to be highly insoluble in water, highly lipophilic and precipitates in the presence of gastrointestinal fluids, therefore is instructed to be taken with food. The lipid component in food enhances its solubilisation, increasing the amount available for absorption, which in turn increases bioavailability.

Lipid-based formulations (LBF) are designed to mimic the food effect by presenting the drug in its solubilised form to the GIT. However, there are still drawbacks with liquid LBFs such as low drug loading capacity and poor stability where the drug recrystalises in an aqueous environment. Therefore, the aims of this study were (1) to fabricate fenofibrate-loaded inulin-lipid hybrid (ILH) formulations with various inulin:lipid ratios, (2) to investigate fenofibrate solubilisation during in vitro dissolution and lipolysis from the fabricated formulations, and (3) to compare ILH formulations to crystalline fenofibrate, commercial product APO-fenofibrate and the precursor emulsion.

### Methods

### Preparation of ILH Particles

Inulin-lipid hybrids were prepared by dissolving fenofibrate (50 mg) (obtained from Sigma-Aldrich, Castle Hill, Australia) in a lipid solution containing soybean lecithin (25 mg) and Capmul PG8 (0.5 g) (obtained from Abitec Pty Ltd, NSW, Australia) followed by the addition of water to form a lipid-in-water emulsion then homogenized via sonication. A 2% w/v aqueous solution of inulin was prepared by dissolving inulin (1 g) in water (50 mL), which was added to the emulsion at varying ratios to achieve inulin:lipid ratios of 25:75, 50:50 and 75:25 (based on relative weight of the two components). The inulin:lipid emulsions were subsequently spray dried to form ILH microparticles under the following conditions: 0.5 mL/min emulsion flow rate, 0.6 m³/min, 200° C. inlet temperature, 100° C. outlet temperature and 100% aspirator setting.

### Physicochemical Characteristics

The surface morphology and particle size of ILH particles were determined using scanning electron microscopy (SEM). The drug crystallinity was measured using differential scanning calorimetry (DSC) by heating a small amount of formulation in an aluminium pan over a temperature range of 20-120° C. Drug content was measured using a solvent extraction technique, where a small amount of formulation was added to acetonitrile and sonicated to ensure 100% extraction, centrifuged to precipitate any undissolved drug and analysed using high-performance liquid chromatography (HPLC).

### In Vitro Dissolution Studies

In vitro dissolution studies were performed for 90 min according to standard methods known in the art. Briefly, samples of formulations were added to temperature-controlled vessels containing dissolution media (0.36% SLS). The solubilisation of the drug in aqueous phase was measured by withdrawing samples and filtering them through a 0.45 μm pore filter at certain time points, diluting then analysing them using HPLC.

### In Vitro Lipolysis Studies

In vitro gastrointestinal lipolysis studies were performed for 90 min using a pH-stat apparatus, where samples of formulation were added to temperature-controlled vessels with gastric media and lipase at pH 1.6 for 30 min, then intestinal media and pancreatin extract at pH 6.5 for 60 min (Joyce et al., 2018, Enhancing the lipase-mediated bioaccessibility of omega-3 fatty acids by microencapsulation of fish oil droplets with porous silica particles, Journal of Functional Foods, 47: 491-502). Fasted state simulated gastric and intestinal fluid, and pancreatin extracts were prepared prior to lipolysis. The solubilisation of the drug in aqueous phase was measured by withdrawing samples at certain time points and adding an enzyme inhibitor, diluting then analysed them using HPLC. The pH was titrated with the addition of NaOH by the pH-stat apparatus which correlates to the amount of digestion.

### Results

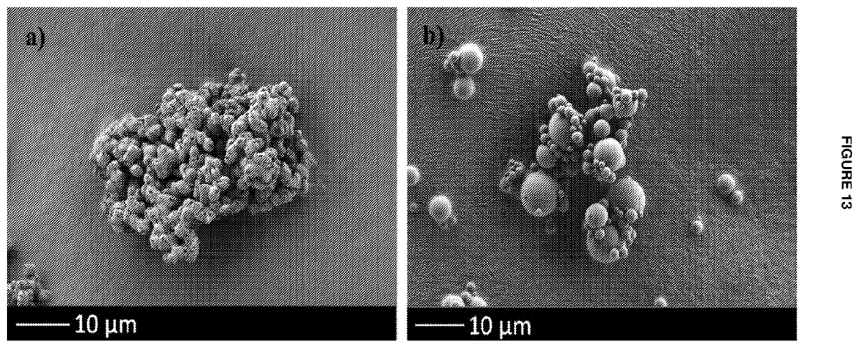

Physicochemical characterisation were analysed and have highlighted the key properties of ILH which may play a major role in impacting drug release, solubilisation, lipolysis, precipitation and stability. The surface morphology and particle size of inulin and ILH particles were examined using SEM imaging (FIG. 13). The aggregation behaviour of inulin is highlighted in the absence and presence of lipid. Spray drying inulin onto the surface of emulsion droplets forms 6-15 μm cohesive units with individual microparticles of various sizes visible within the aggregates (FIG. 13b).

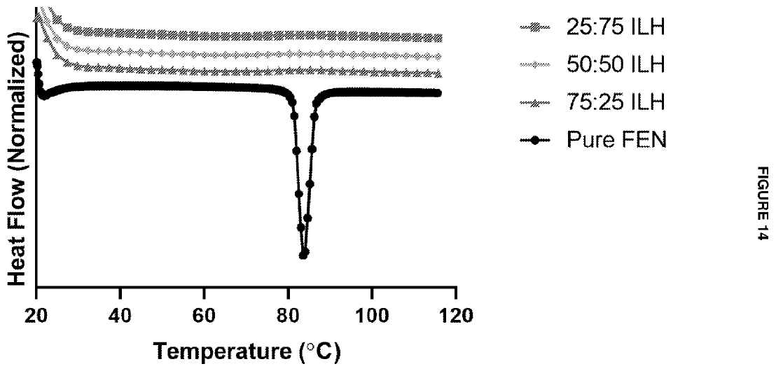

Measuring the crystallinity of drug in each formulation was examined using DSC (FIG. 14). The lack of endothermic peaks from the ILH formulations represent non-crystalline form, compared to the sharp endothermic peak produced by crystalline fenofibrate at 80° C., which corresponds to the melting point of fenofibrate in the crystalline form (Zhang H et al., 2014, Pharmaceutical and pharmacokinetic characteristics of different types of fenofibrate nanocrystals prepared by different bottom-up approaches, Drug Delivery, 21(8): 588-594). The formulations were prepared two weeks earlier and no peaks were reported meaning the formulations remained stable for this period of time.

The drug loads for the formulations were determined using the solvent extraction technique (Table 1). The measured drug loads for 50:50 ILH and 75:25 ILH were similar to the maximum drug loads with high entrapment efficiencies of 84.2% and 80%, respectively. To get equivalent doses for administration, the amount of 75:25 ILH has to be doubled. The higher maximum drug load for 25:75 ILH resulted in a measured drug load similar to 50:50 ILH with an entrapment efficiency of 52.6%.

TABLE 1
Drug content of ILH formulations based
on 80% saturation solubility

| | Maximum | Measured | Entrapment |
|---|---|---|---|
| | drug load | drug load | efficiency |
| Formulation | (% w/w) | (% w/w) | (%) |
| 50:50 ILH | 3.8 | 3.2 | 84.2 |
| 75:25 ILH | 2 | 1.6 | 80 |
| 25:75 ILH | 5.7 | 3 | 52.6 |

In vitro studies were dosed equivalent to 3 mg fenofibrate for lipolysis and 10 mg for dissolution based on the measured drug load in each formulation.

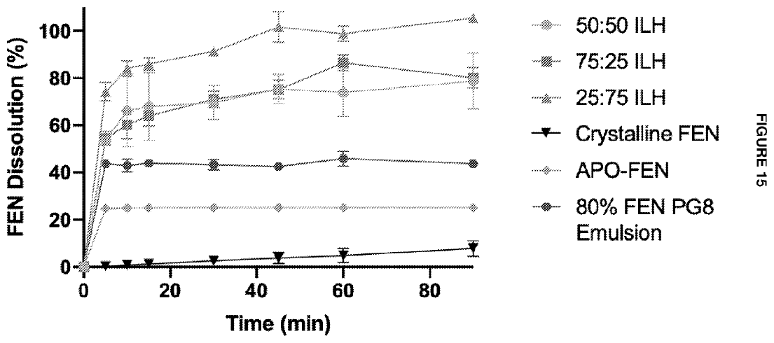

In vitro studies were utilised to investigate the dissolution, solubilisation and digestion behaviour of the ILH formulations in a simulated human gut under digesting and non-digesting conditions. In vitro dissolution was performed to measure the percentage drug released from each formulation under non-digesting conditions (FIG. 15). The results demonstrate 4-6 fold greater fenofibrate dissolution achieved by all ILH formulations than crystalline, commercial and emulsified fenofibrate, with 100% dissolution for 25:75 ILH and 80% dissolution for 50:50 ILH and 75:25 ILH at 90 minutes.

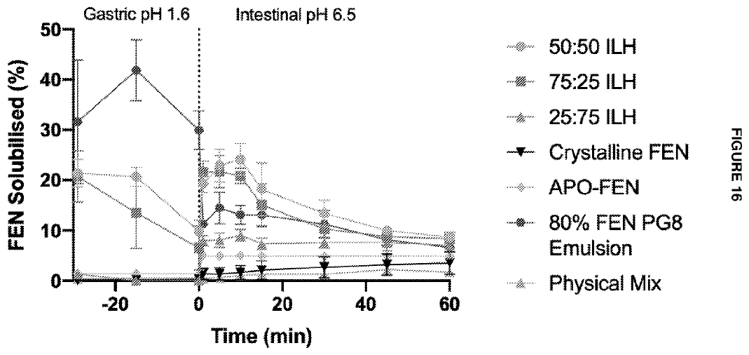

In vitro lipolysis was performed to measure the aqueous solubilisation of each formulation during a two-phase gastrointestinal digestion (FIG. 16). The crystalline drug, commercial drug and physical mix exhibit low solubilisation of 1.7%-5% after 90 min. Delivering fenofibrate in the emulsion reveals the aqueous solubilisation is much higher during the gastric phase, however significant intestinal precipitation occurs with a 2.7-fold decline in the percent of fenofibrate solubilised. The ILH formulations show an opposite trend to the emulsion where significant enhancement in aqueous solubilisation occurs triggered by the transition into the intestinal phase with 50:50 ILH measuring the highest concentration of 24.2% at 10 min intestinal phase.

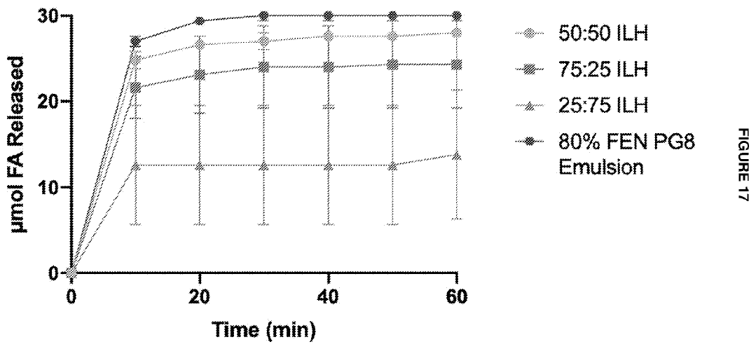

The amount of lipid digestion can be measured during the intestinal phase of in vitro lipolysis as the fatty acids produced are neutralised by titrating NaOH in order to maintain pH throughout the study (FIG. 17). 50:50 ILH, 75:25 ILH and the emulsion show high intestinal lipid digestion from the high fatty acid release with no significant differences between these formulations. However, an observable difference was measured with 25:75 ILH with a 2-fold decrease in lipid digestion at 60 min compared to 50:50 ILH.

### DISCUSSION

Inulin-lipid hybrids were fabricated to achieve a free-flowing powder of various inulin:lipid ratios of 50:50, 75:25 and 25:75. All fabricated formulations appeared to be stable for two weeks evident by DSC data (FIG. 14). The high entrapment efficiencies as shown in Table 1, of 84.2% and 80% for 50:50 ILH and 75:25 ILH, suggest inulin is a more beneficial drug carrier than previously discovered carriers including montmorillonite where the entrapment efficiency was 63.4% (Denning T J et al., 2017, Montmorillonite-lipid hybrid carriers for ionizable and neutral poorly water-soluble drugs: Formulation, characterization and in vitro lipolysis studies, International Journal of Pharmaceutics, 526: 95-105). The drug load of 75:25 ILH was half of the drug load retained by 50:50 ILH indicating a 2× increase in formulation is required to achieve equivalent doses, thus being a limitation from a formulation perspective. The low entrapment efficiency of 52.6% for 25:75 ILH suggest either inulin has a maximum lipid loading capability or the incomplete lipid loading is thought to be through the fabrication process, where the excess lipid is not coated by the inulin and adheres to the wall of the spray dryer.

Both 50:50 ILH and 75:25 ILH formulations achieve comparable performance during in vitro dissolution and lipolysis (FIGS. 15 and 16). However, 50:50 ILH achieves a 2-fold increase in drug load which is desirable in manufacturing. Despite the enhanced performance of 75:25 ILH during in vitro dissolution studies, under digesting conditions it displays poor performance. The low drug loading efficiency suggests the possibility of lipid loss during the spray drying process which may have contributed to the decrease in performance. Despite precipitation of fenofibrate during in vitro lipolysis, all ILH formulations significantly enhance its solubilisation when compared to pure drug, APO-fenofibrate and the precursor emulsion. Therefore, inulin is an important component in preventing precipitation of fenofibrate and has the potential to eliminate the rate-limiting dissolution step to absorption.

The increase in solubilisation from gastric to intestinal lipolysis of all ILH formulations suggest inulin retains its stability through the gastric phase and as the LBF is released from inulin it retains fenofibrate in its presolubilised form (FIG. 16). The significantly higher solubilisation during the intestinal phase for 50:50 ILH and 75:25 ILH suggest the inulin contributes to a delayed release by controlling lipase mediated action and accessibility resulting in much less precipitation compared to the opposing trend shown by the emulsion. When orally administered, the lipid component of the ILH formulation is digested and forms a lipophilic environment for the drug to remain solubilised, leading to greater dissolution and absorption for fenofibrate. The 2-fold decrease of 25:75 ILH compared to 50:50 ILH in FIG. 17 suggests the lower ratio of inulin to lipid creates larger particles composed of a higher proportion of lipid, where a smaller surface area is available for lipid digestion. Therefore, when designing an ILH, the inulin:lipid ratio is an important parameter that can influence their performance.

Fenofibrate precipitation behaviour from LBFs has been previously reported by multiple studies by the loss of solubilisation capacity once digested resulting in drug precipitation. This behaviour did not correlate with poor performance in vivo, where despite precipitation occurring, a rapid rate of re-dissolution and absorption was aided by the formation of colloidal species from the LBFs. The incorporation of fenofibrate in a LBF resulted in enhanced performance compared to commercial fenofibrate. For a highly permeable drug such as fenofibrate, its precipitation is expected to be less profound in vivo.

### Conclusion

Throughout this study three different ratios of inulin to lipid were investigated and results demonstrate that the performance of 50:50 ILH and 75:25 ILH showed significant benefit in the dissolution and solubilisation of fenofibrate. This suggests that in the presence of lipid, the rate-limiting step to dissolution can be removed and with the addition of inulin, the residence time of fenofibrate in solution is increased where it is available for absorption.

The increasing number of poorly water-soluble drugs (PWSDs) makes it difficult to design innovative formulations in order to overcome their inherent slow dissolution and poor absorption. When co-administering these drugs with food, the bioavailability is enhanced due to the lipid component causing self-emulsification where the drugs can be solubilised within. To remove this food effect, formulation scientists have developed lipid-based formulations (LBFs) to present the drug in an already presolubilised state to the GIT. LBFs have had promising results, however due to their limitations of poor stability, costly manufacturing and poor correlation between in vitro and in vivo studies, only 37 commercially available LBFs in the liquid-state are currently in market. Solid-state LBFs have gained substantial attention and have been proven to overcome these limitations. Furthermore, according to research, they have shown promising results in enhancing the bioavailability of PWSDs. However, there has been no solid-state LBFs commercialised to date. During this study we have designed a solid-state LBF and were able to demonstrate its ability in enhancing the solubilisation of a model PWSD that outperformed a commercially available dosage form. This formulation strategy can be applied to other more clinically relevant compounds.

### Example 4

### Composition for Improving Health

In this study, the influence of polysaccharide-lipid hybrids in enhancing the health benefits of subjects was investigated. The polysaccharide-lipid hybrids were administered in the absence of an active substance or drug. In this instance, a rat anti-obesity model (where all groups were fed a high fat diet to induce weight gain) were dosed with inulin-lipid hybrids (ILH) on their own (i.e. no other drug or active substance present) for a period of 3 weeks.

### Methods

### In Vivo Pharmacodynamic Study

Four-week-old male Sprague-Dawley rats were housed in groups of two in a temperature-, humidity-, and pressure-controlled, animal holding facility with a 12 h/12 h light/dark cycle. Following a one-week acclimatisation period, rats were placed on a high-fat diet (HFD; 22% fat/44% energy from fat) and were separated into the following treatment groups (n=6): control group (PBS; 10 mL/kg), and ILH microparticles (1 g/kg). ILH was dispersed in PBS (10 mL/kg) prior to daily administration between 17:00 and 19:00 via oral gavage for a period of 21 days. Evening dosing was selected for this study in an attempt to simulate rodents natural behaviour, since they are most active and eat mostly at night. Rodent body weight was measured daily, immediately prior to dosing.

### Sample Collection and Analysis

After 21 days of dosing, rats were fasted for 24 h with free access to water prior to being anaesthesized with (200 mg/kg) ilium sodium pentobarbital (60 mg/mL). Blood samples were collected via cardiac puncture. Blood triglycerides, high-density lipoprotein (HDL) and blood glucose levels were analyzed using a CardioChek PA Analyzer (Pts Diagnostics). HbA1c levels were analyzed using A1CNow+ (Bayer). Fresh fecal samples were combined with cecal contents (following humane killing) and sent for 16S rRNA sequencing at the Australian Genomics Research Facility (Brisbane, Australia). 16S rRNA sequences were processed and analyzed using Quantitative Insights into Microbiology Ecology (QIIME 1.8). Raw reads were imported into the Qiagen CLC Genomics Workbench for all post-hoc analyses.

### Results

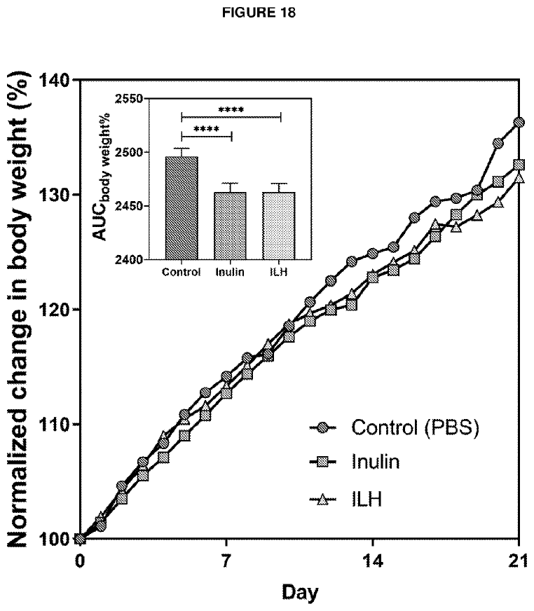

Rodent weight gain was monitored daily over the study period and was normalized based on the initial body weight, since each animal started the study at varying weights. After 21 days of exposure to a HFD, the control group gained 136±12% of their original body weight (FIG. 18). Supplementation of the HFD with both ILH microparticles induced a statistically significant reduction in body weight gain (131±2.6%), compared to the control group. This was comparable to the reduction in weight gain induced by inulin (132±3.2%). Calculation of area-under-the-curve (AUC_{body weight %}) reveals that both treatment groups (i.e. inulin and ILH microparticles) exerted statistically significant differences in AUC_{body weight %} compared to the control group. There was no statistical significance in AUC_{body weight %} between the inulin and ILH treatment groups.

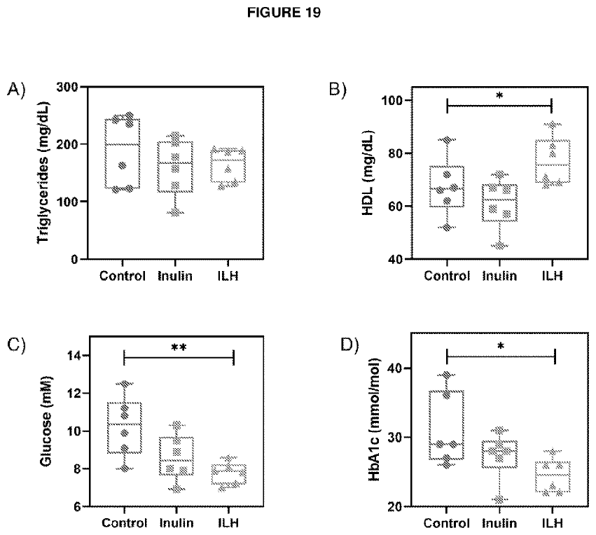

Treatment-induced changes to biomarkers related to metabolic risk factors are presented in FIG. 19, which include blood triglyceride, HDL, glucose and HbA1c levels. ILH microparticles were shown to induce positive changes to blood lipid levels, compared to both the control group and the inulin treatment group. This is evidenced through a greater reduction in blood triglyceride levels and a statistically significant increase in HDL (i.e. ‘good’ cholesterol) levels. Further, ILH microparticles provided a statistically significant reduction in blood glucose levels, which correlated well with a statistically significant reduction in HbA1c levels. Thus, these findings suggest a synergistic effect between the inulin and medium chain triglycerides in promoting positive metabolic health through regulation of key biomarkers linked to the development of metabolic syndrome/obesity.

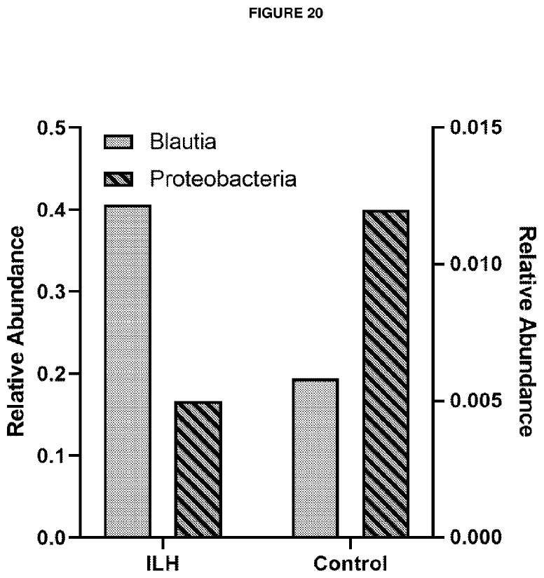

Microbiome analysis following the 21-day study highlighted that ILH microparticles increase the relative abundance of the health-promoting bacteria, Blautia, and reduced the relative abundance of the pathogenic bacteria, Proteobacteria, when compared to the control group (FIG. 20). This suggests that ILH can be employed as a therapeutic to improve gut health by positively modulating the gut microbiome.

### DISCUSSION

Treating rodents exposed to a high-fat diet with ILH microparticles was shown to promote metabolic health through the reduction in weight gain and positive changes in key biomarkers linked with a heightened risk for developing metabolic syndrome/obesity. Combining inulin with medium chain triglycerides, through the formation of ILH microparticles, was not shown to induce a greater reduction in weight gain compared to inulin alone; however, the inclusion of medium chain triglycerides within this hybrid formulation was shown to significantly promote positive changes in key metabolic biomarkers, including blood triglycerides, HDL, glucose and HbA1c levels. This suggests that ILH microparticles provide a multifunctional mechanism of action, compared to inulin alone, and thus should be pursued further as a pharmaceutical agent used for treating or preventing obesity.

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

It is to be noted that where a range of values is expressed, it will be clearly understood that this range encompasses the upper and lower limits of the range, and all numerical values or sub-ranges in between these limits as if each numerical value and sub-range is explicitly recited. The statement “about X % to Y %” has the same meaning as “about X % to about Y %,” unless indicated otherwise.

The term “about” as used in the specification means approximately or nearly and in the context of a numerical value or range set forth herein is meant to encompass variations of +/−10% or less, +/−5% or less, +/−1% or less, or +/−0.1% or less of and from the numerical value or range recited or claimed.

It is also to be noted that, as used herein, the singular forms “a”, “an” and “the” include plural aspects unless the context already dictates otherwise.

The subject headings used herein are included only for the ease of reference of the reader and should not be used to limit the subject matter found throughout the disclosure or the claims. The subject headings should not be used in construing the scope of the claims or the claim limitations.

The description provided herein is in relation to several embodiments which may share common characteristics and features. It is to be understood that one or more features of one embodiment may be combinable with one or more features of the other embodiments. In addition, a single feature or combination of features of the embodiments may constitute additional embodiments.

All methods described herein can be performed in any suitable order unless indicated otherwise herein or clearly contradicted by context. The use of any and all examples, or exemplary language (e.g., “such as”) provided herein, is intended merely to better illuminate the example embodiments and does not pose a limitation on the scope of the claimed invention unless otherwise claimed. No language in the specification should be construed as indicating any non-claimed element as essential.

It will be apparent to the person skilled in the art that while the invention has been described in some detail for the purposes of clarity and understanding, various modifications and alterations to the embodiments and methods described herein may be made without departing from the scope of the inventive concept disclosed in this specification.

Those skilled in the art will appreciate that the invention described herein is susceptible to variations and modifications other than those specifically described. It is to be understood that the invention includes all such variations and modifications. The invention also includes all of the steps, features, compositions and compounds referred to, or indicated in this specification, individually or collectively, and any and all combinations of any two or more of the steps or features.

Finally, reference is made to standard textbooks of molecular biology that contain methods for carrying out basic techniques encompassed by the present invention. See, for example, Green M R and Sambrook J, 2012, supra.

Future patent applications may be filed in Australia or overseas on the basis of the present application, for example by claiming priority from the present application, by claiming a divisional status and/or by claiming a continuation status. It is to be understood that the following claims are not intended to limit the scope of what may be claimed in any such future application. Furthermore, the claims should not be considered to limit the understanding of (or exclude other understandings of) the invention inherent in the present disclosure. Features may be added to or omitted from the claims at a later date, so as to further define the invention.

1. A dry composition comprising:
(i) a polysaccharide; and
(ii) lipid droplets,
wherein the lipid droplets are encapsulated within polymeric chains of the polysaccharide, and wherein the polysaccharide is not in a nano-particulate form.

2. The composition of claim 1, wherein the polysaccharide is a dietary polysaccharide.

3. The composition of claim 2, wherein the polysaccharide is selected from the group consisting of inulin, cellulose and glucomannan.

4. The composition of claim 3, wherein the polysaccharide is inulin.

5. The composition of any one of claims 1 to 4, wherein the lipid droplets comprise a medium chain length fatty acid.

6. The composition of any one of claims 1 to 5, wherein the ratio of polysaccharide to lipid in the composition ranges from about 10:90 to about 90:10.

7. The composition of claim 6, wherein the ratio of polysaccharide to lipid in the composition ranges from about 50:50 to about 75:25.

8. The composition of claim 4, wherein the composition is used for improving the health of a subject.

9. The composition of claim 8, wherein the gastrointestinal health of the subject is improved.

10. The composition of any one of claims 1 to 9, further comprising an active substance, wherein the active substance is contained within the lipid droplets.

11. The composition of claim 10, wherein the active substance is an agent that can dissolve in the lipid droplets.

12. The composition of claim 10 or claim 11, wherein the active substance is a pharmaceutical agent.

13. The composition of claim 12, wherein the pharmaceutical agent is a poorly water-soluble drug.

14. The composition of claim 12 or claim 13, wherein the pharmaceutical agent is selected from the group consisting of an antimicrobial agent, an anti-inflammatory agent, an anti-histamine, a cholesterol-lowering drug, or a psychotropic drug.

15. The composition of claim 14, wherein the antimicrobial agent is one or more of an antibiotic, an antimicrobial peptide, and an antifungal agent.

16. The composition of claim 15, wherein the antibiotic is selected from the group consisting of rifampicin, tobramycin and vancomycin.

17. The composition of claim 14, wherein the cholesterol-lowering drug is a statin or fenofibrate.

18. The composition of claim 17, wherein the statin is simvastatin.

19. The composition of claim 14, wherein the psychotropic drug is lurasidone.

20. The composition of any one of claim 1 to 19, further comprising an excipient or stabilizer.

21. The composition of claim 20, wherein the stabilizer is lecithin.

22. The composition of any one of claims 1 to 21, wherein the composition is formulated for oral delivery.

23. The composition of any one of claims 1 to 21, wherein the composition is produced by a method comprising:
(i) producing a lipid-in-water nano-emulsion comprising the lipid droplets and the polysaccharide, wherein the polysaccharide is dissolved in the aqueous phase of the nano-emulsion; and
(ii) spray-drying the nano-emulsion.

24. The composition of claim 23, wherein the nano-emulsion is produced by homogenizing a mixture comprising the lipid droplets and adding the polysaccharide in aqueous form to the mixture.

25. The composition of any one of claims 1 to 24, wherein the microparticles have an average diameter of <15 μm.

26. A method for improving the health of a subject, or for treating or preventing a disease or disorder in a subject, the method comprising administering the composition of any one of claims 4 to 25 to the subject.

27. The method of claim 26, wherein the gastrointestinal health of the subject is improved.

28. The method of claim 26, wherein a metabolic disease or disorder is treated or prevented in the subject.

29. A method for treating or preventing a microbial infection is a subject, the method comprising administering the composition of any one of claims 12 to 25 to the subject, wherein the pharmaceutical agent is an antimicrobial agent.

30. The method of claim 29, wherein the microbial infection is a bacterial infection.

31. The method of claim 30, wherein the bacterial infection is due to Staphylococcus aureus.

32. The method of claim 30 or claim 31, wherein the bacterial infection forms part of a biofilm.

33. A method for administering an active substance to a subject, wherein the method comprises administering to the subject a composition of any one of claims 10 to 25.

34. A method for producing a composition of any one of claims 1 to 33, wherein the method comprises spray-drying a lipid-in-water nano-emulsion comprising the lipid droplets and the polysaccharide.

35. A dry composition comprising:
(i) a polysaccharide;
(ii) lipid droplets; and
(iii) an excipient or stabilizer,
wherein the lipid droplets are encapsulated within polymeric chains of the polysaccharide, and wherein the polysaccharide is not in a nano-particulate form.

36. The composition of claim 35, wherein the polysaccharide is a dietary polysaccharide.

37. The composition of claim 36, wherein the polysaccharide is selected from the group consisting of inulin, cellulose and glucomannan.

38. The composition of claim 37, wherein the polysaccharide is inulin.

39. The composition of any one of claims 35 to 38, wherein the lipid droplets comprise a medium chain length fatty acid.

40. The composition of any one of claims 35 to 39, wherein the ratio of polysaccharide to lipid in the composition ranges from about 10:90 to about 90:10.

41. The composition of claim 40, wherein the ratio of polysaccharide to lipid in the composition ranges from about 50:50 to about 75:25.

42. The composition of any one of claims 35 to 41, wherein the stabilizer is lecithin.

43. The composition of any one of claims 38 to 42, wherein the composition is used for improving the health of a subject.

44. The composition of claim 43, wherein the gastrointestinal health of the subject is improved.

45. The composition of any one of claims 35 to 44, further comprising an active substance, wherein the active substance is contained within the lipid droplets.

46. The composition of claim 45, wherein the active substance is an agent that can dissolve in the lipid droplets.

47. The composition of claim 45 or claim 46, wherein the active substance is a pharmaceutical agent.

48. The composition of claim 47, wherein the pharmaceutical agent is a poorly water-soluble drug.

49. The composition of claim 47 or claim 48, wherein the pharmaceutical agent is selected from the group consisting of an antimicrobial agent, an anti-inflammatory agent, an anti-histamine, a cholesterol-lowering drug, or a psychotropic drug.

50. The composition of claim 49, wherein the antimicrobial agent is one or more of an antibiotic, an antimicrobial peptide, and an antifungal agent.

51. The composition of claim 50, wherein the antibiotic is selected from the group consisting of rifampicin, tobramycin and vancomycin.

52. The composition of claim 49, wherein the cholesterol-lowering drug is a statin or fenofibrate.

53. The composition of claim 52, wherein the statin is simvastatin.

54. The composition of claim 49, wherein the psychotropic drug is lurasidone.

55. The composition of any one of claims 35 to 54, wherein the composition is produced by a method comprising:
(i) producing a lipid-in-water nano-emulsion comprising the lipid droplets, the polysaccharide, and the excipient or stabilizer, wherein the polysaccharide is dissolved in the aqueous phase of the nano-emulsion; and
(ii) spray-drying the nano-emulsion.

56. The composition of claim 55, wherein the nano-emulsion is produced by homogenizing a mixture comprising the lipid droplets and the excipient or stabilizer, and adding the polysaccharide in aqueous form to the mixture.

57. The composition of any one of claims 35 to 56, wherein the microparticles have an average diameter of <15 μm.

58. A method for improving the health of a subject, or for treating or preventing a disease or disorder in a subject, the method comprising administering the composition of any one of claims 38 to 57 to the subject.

59. The method of claim 58, wherein the gastrointestinal health of the subject is improved.

60. The method of claim 58, wherein a metabolic disease or disorder is treated or prevented in the subject.

61. A method for treating or preventing a microbial infection is a subject, the method comprising administering the composition of any one of claims 47 to 57 to the subject, wherein the pharmaceutical agent is an antimicrobial agent.

62. The method of claim 61, wherein the microbial infection is a bacterial infection.

63. The method of claim 62, wherein the bacterial infection is due to Staphylococcus aureus.

64. The method of claim 62 or claim 63, wherein the bacterial infection forms part of a biofilm.

65. A method for administering an active substance to a subject, wherein the method comprises administering to the subject a composition of any one of claims 45 to 57.

66. A method for producing a composition of any one of claims 35 to 57, wherein the method comprises spray-drying a lipid-in-water nano-emulsion comprising the lipid droplets, the polysaccharide, and the excipient or stabilizer.

67. A dry composition comprising:
(i) inulin;
(ii) an active substance; and
(iii) lipid droplets,
wherein the lipid droplets are encapsulated within polymeric chains of inulin, and wherein the polysaccharide is not in a nano-particulate form.

68. The composition of claim 67, wherein the lipid droplets comprise a medium chain length fatty acid.

69. The composition of claim 67 or claim 68, wherein the ratio of inulin to lipid in the composition ranges from about 10:90 to about 90:10.

70. The composition of claim 69, wherein the ratio of inulin to lipid in the composition ranges from about 50:50 to about 75:25.

71. The composition of any one of claims 67 to 70, wherein the active substance is contained within the lipid droplets.

72. The composition of claim 71, wherein the active substance is an agent that can dissolve in the lipid droplets.

73. The composition of any one of claims 67 to 72, wherein the active substance is a pharmaceutical agent.

74. The composition of claim 73, wherein the pharmaceutical agent is a poorly water-soluble drug.

75. The composition of claim 73 or claim 74, wherein the pharmaceutical agent is selected from the group consisting of an antimicrobial agent, an anti-inflammatory agent, an anti-histamine, a cholesterol-lowering drug, or a psychotropic drug.

76. The composition of claim 75, wherein the antimicrobial agent is one or more of an antibiotic, an antimicrobial peptide, and an antifungal agent.

77. The composition of claim 76, wherein the antibiotic is selected from the group consisting of rifampicin, tobramycin and vancomycin.

78. The composition of claim 75, wherein the cholesterol-lowering drug is a statin or fenofibrate.

79. The composition of claim 78, wherein the statin is simvastatin.

80. The composition of claim 75, wherein the psychotropic drug is lurasidone.

81. The composition of any one of claim 67 to 80, further comprising an excipient or stabilizer.

82. The composition of claim 81, wherein the stabilizer is lecithin.

83. The composition of any one of claims 67 to 82, wherein the composition is formulated for oral delivery.

84. The composition of any one of claims 67 to 83, wherein the composition is produced by a method comprising:
(i) producing a lipid-in-water nano-emulsion comprising the lipid droplets, the active substance, and the inulin, wherein the inulin is dissolved in the aqueous phase of the nano-emulsion; and
(ii) spray-drying the nano-emulsion.

85. The composition of claim 84, wherein the nano-emulsion is produced by homogenizing a mixture comprising the lipid droplets and the active substance, and adding the inulin in aqueous form to the mixture.

86. The composition of any one of claims 67 to 85, wherein the microparticles have an average diameter of <15 μm.

87. A method for treating or preventing a disease or disorder in a subject, the method comprising administering the composition of any one of claims 67 to 86 to the subject.

88. A method for treating or preventing a microbial infection in a subject, the method comprising administering the composition of any one of claims 73 to 86 to the subject, wherein the pharmaceutical agent is an antimicrobial agent.

89. The method of claim 88, wherein the microbial infection is a bacterial infection.

90. The method of claim 89, wherein the bacterial infection is due to Staphylococcus aureus.

91. The method of claim 89 or claim 90, wherein the bacterial infection forms part of a biofilm.

92. A method for administering an active substance to a subject, wherein the method comprises administering to the subject a composition of any one of claims 67 to 86.

93. A method for producing a composition of any one of claims 67 to 86, wherein the method comprises spray-drying a lipid-in-water nano-emulsion comprising the lipid droplets, the active substance, and the inulin.

94. A dry composition comprising:
(i) inulin;
(ii) lipid droplets; and
(iii) lecithin,
wherein the lipid droplets are encapsulated within polymeric chains of the inulin, and wherein the inulin is not in a nano-particulate form.

95. A method for improving the gastrointestinal health of a subject, the method comprising administering to the subject a dry composition comprising:
(i) inulin;
(ii) lipid droplets; and
(iii) lecithin,
wherein the lipid droplets are encapsulated within polymeric chains of the inulin, and wherein the inulin is not in a nano-particulate form.

96. A method for treating or preventing a metabolic disorder or disease in a subject, the method comprising administering to the subject a dry composition comprising:
(i) inulin;
(ii) lipid droplets; and
(iii) lecithin,
wherein the lipid droplets are encapsulated within polymeric chains of the inulin, and wherein the inulin is not in a nano-particulate form.

97. A dry composition comprising:
(i) inulin;
(ii) lipid droplets; and
(iii) a poorly water-soluble active substance,
wherein the lipid droplets are encapsulated within polymeric chains of the inulin, and wherein the inulin is not in a nano-particulate form.

98. A method for treating or preventing a disease or disorder in a subject, the method comprising administering to the subject a dry composition comprising:
(i) inulin;
(ii) lipid droplets; and
(iii) a poorly water-soluble active substance,
wherein the lipid droplets are encapsulated within polymeric chains of the inulin, and wherein the inulin is not in a nano-particulate form.

99. A dry composition comprising:
(i) inulin;
(ii) lipid droplets; and
(iii) an antibiotic,
wherein the lipid droplets are encapsulated within polymeric chains of the inulin, and wherein the inulin is not in a nano-particulate form.

100. A method for treating or preventing a bacterial infection in a subject, the method comprising administering to the subject a dry composition comprising:
(i) inulin;
(ii) lipid droplets; and
(iii) an antibiotic,
wherein the lipid droplets are encapsulated within polymeric chains of the inulin, and wherein the inulin is not in a nano-particulate form.
